SEQUENCE LISTING (30 sequences)

SEQ ID NO: 1 - protein, 5 aa, synthetic construct
  REGION 1..5: note = source = /note="Description of Artificial Sequence: Synthetic peptide"
TYTMS

SEQ ID NO: 2 - protein, 18 aa, synthetic construct
  REGION 1..18: note = source = /note="Description of Artificial Sequence: Synthetic peptide"
TISPGDSFGYYYPDSVQG

SEQ ID NO: 3 - protein, 12 aa, synthetic construct
  REGION 1..12: note = source = /note="Description of Artificial Sequence: Synthetic peptide"
DIYYNYGAWFAY

SEQ ID NO: 4 - protein, 15 aa, synthetic construct
  REGION 1..15: note = source = /note="Description of Artificial Sequence: Synthetic peptide"
KASQSVDYDGDSYMN

SEQ ID NO: 5 - protein, 7 aa, synthetic construct
  REGION 1..7: note = source = /note="Description of Artificial Sequence: Synthetic peptide"
AASTLES

SEQ ID NO: 6 - protein, 9 aa, synthetic construct
  REGION 1..9: note = source = /note="Description of Artificial Sequence: Synthetic peptide"
QQANEDPRT

SEQ ID NO: 7 - protein, 122 aa, synthetic construct
  REGION 1..122: note = source = /note="Description of Artificial Sequence: Synthetic polypeptide"
DVQLVESGGGLVKPGGSLRLSCAASGFTFSTYTMSWVRQAPGKGLEWVATISPGDSFGYYYPDSVQGRFTISRDNAKNSLYLQMNSLRAEDTAVYYCTRDIYYNYGAWFAYWGQGTLVTVSS

SEQ ID NO: 8 - protein, 111 aa, synthetic construct
  REGION 1..111: note = source = /note="Description of Artificial Sequence: Synthetic polypeptide"
DIQLTQSPSSLSASVGDRVTITCKASQSVDYDGDSYMNWYQQKPGKAPKLLIYAASTLESGVPSRFSGSGSGTDFTLTISSLQPEDFATYYCQQANEDPRTFGQGTKVEIK

SEQ ID NO: 9 - protein, 451 aa, synthetic construct
  REGION 1..451: note = source = /note="Description of Artificial Sequence: Synthetic polypeptide"
DVQLVESGGGLVKPGGSLRLSCAASGFTFSTYTMSWVRQAPGKGLEWVATISPGDSFGYYYPDSVQGRFTISRDNAKNSLYLQMNSLRAEDTAVYYCTRDIYYNYGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 218 aa, synthetic construct
  REGION 1..218: note = source = /note="Description of Artificial Sequence: Synthetic polypeptide"
DIQLTQSPSSLSASVGDRVTITCKASQSVDYDGDSYMNWYQQKPGKAPKLLIYAASTLESGVPSRFSGSGSGTDFTLTISSLQPEDFATYYCQQANEDPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 7 aa, synthetic construct
  REGION 1..7: note = source = /note="Description of Artificial Sequence: Synthetic peptide"
GFTFSTY

SEQ ID NO: 12 - protein, 7 aa, synthetic construct
  REGION 1..7: note = source = /note="Description of Artificial Sequence: Synthetic peptide"
SPGDSFG

SEQ ID NO: 13 - protein, 12 aa, synthetic construct
  REGION 1..12: note = source = /note="Description of Artificial Sequence: Synthetic peptide"
DIYYNYGAWFAY

SEQ ID NO: 14 - protein, 15 aa, synthetic construct
  REGION 1..15: note = source = /note="Description of Artificial Sequence: Synthetic peptide"
KASQSVDYDGDSYMN

SEQ ID NO: 15 - protein, 7 aa, synthetic construct
  REGION 1..7: note = source = /note="Description of Artificial Sequence: Synthetic peptide"
AASTLES

SEQ ID NO: 16 - protein, 9 aa, synthetic construct
  REGION 1..9: note = source = /note="Description of Artificial Sequence: Synthetic peptide"
QQANEDPRT

SEQ ID NO: 17 - protein, 10 aa, synthetic construct
  REGION 1..10: note = source = /note="Description of Artificial Sequence: Synthetic peptide"
GFTFSTYTMS

SEQ ID NO: 18 - protein, 11 aa, synthetic construct
  REGION 1..11: note = source = /note="Description of Artificial Sequence: Synthetic peptide"
TISPGDSFGYY

SEQ ID NO: 19 - protein, 12 aa, synthetic construct
  REGION 1..12: note = source = /note="Description of Artificial Sequence: Synthetic peptide"
DIYYNYGAWFAY

SEQ ID NO: 20 - protein, 15 aa, synthetic construct
  REGION 1..15: note = source = /note="Description of Artificial Sequence: Synthetic peptide"
KASQSVDYDGDSYMN

SEQ ID NO: 21 - protein, 7 aa, synthetic construct
  REGION 1..7: note = source = /note="Description of Artificial Sequence: Synthetic peptide"
AASTLES

SEQ ID NO: 22 - protein, 9 aa, synthetic construct
  REGION 1..9: note = source = /note="Description of Artificial Sequence: Synthetic peptide"
QQANEDPRT

SEQ ID NO: 23 - protein, 6 aa, synthetic construct
  REGION 1..6: note = source = /note="Description of Artificial Sequence: Synthetic peptide"
STYTMS

SEQ ID NO: 24 - protein, 14 aa, synthetic construct
  REGION 1..14: note = source = /note="Description of Artificial Sequence: Synthetic peptide"
WVATISPGDSFGYY

SEQ ID NO: 25 - protein, 13 aa, synthetic construct
  REGION 1..13: note = source = /note="Description of Artificial Sequence: Synthetic peptide"
TRDIYYNYGAWFA

SEQ ID NO: 26 - protein, 11 aa, synthetic construct
  REGION 1..11: note = source = /note="Description of Artificial Sequence: Synthetic peptide"
DYDGDSYMNWY

SEQ ID NO: 27 - protein, 10 aa, synthetic construct
  REGION 1..10: note = source = /note="Description of Artificial Sequence: Synthetic peptide"
LLIYAASTLE

SEQ ID NO: 28 - protein, 8 aa, synthetic construct
  REGION 1..8: note = source = /note="Description of Artificial Sequence: Synthetic peptide"
QQANEDPR

SEQ ID NO: 29 - protein, 213 aa, Homo sapiens
MVPEEEPQDREKGLWWFQLKVWSMAVVSILLLSVCFTVSSVVPHNFMYSKTVKRLSKLREYQQYHPSLTCVMEGKDIEDWSCCPTPWTSFQSSCYFISTGMQSWTKSQKNCSVMGADLVVINTREEQDFIIQNLKRNSSYFLGLSDPGGRRHWQWVDQTPYNENVTFWHSGEPNNLDERCAIINFRSSEEWGWNDIHCHVPQKSICKMKKIYI

SEQ ID NO: 30 - protein, 86 aa, Homo sapiens
MIPAVVLLLLLLVEQAAALGEPQLCYILDAILFLYGIVLTLLYCRLKIQVRKAAITSYEKSDGVYTGLSTRNQETYETLKHEKPPQ

(19) United States
(12) Patent Application Publication
(10) Pub. No.: US 2025/0289895 A1
(43) Pub. Date: Sep. 18, 2025

(54) PHARMACEUTICAL COMPOSITIONS AND DOSAGE REGIMENS FOR CLINICAL USE OF ANTI-BLOOD DENDRITIC CELL ANTIGEN 2 ANTIBODIES

(71) Applicant: Biogen MA Inc., Cambridge, MA (US)

(72) Inventors: Mark R.H. Krebs, Arlington, MA (US); David Dai, Chestnut Hill, MA (US); Shantanu Sule, Arlington, MA (US); Dania Mounir Rabah, Cambridge, MA (US); David Martin, Brookline, MA (US)

(21) Appl. No.: 19/060,474

(22) Filed: Feb. 21, 2025

Related U.S. Application Data
(63) Continuation of application No. 18/737,724, filed on Jun. 7, 2024, now abandoned.
Application No. 18/737,724 is a continuation of application No. 16/095,475, filed on Oct. 22, 2018, now abandoned.
(60) Provisional application No. 62/328,959, filed on Apr. 28, 2016.

(51) Int. Cl. C07K 16/28; A61K 9/00; A61K 39/00; A61P 37/06

(52) U.S. Cl. CPC C07K 16/2851; A61K 9/0019; A61P 37/06; A61K 2039/545

(57) ABSTRACT

Formulations and dosage regimens of anti-Blood Dendritic Cell Antigen 2 (BDCA2) antibodies are provided. These formulations and dosage regimens find use in the treatment of BDCA2-associated disorders such as systematic lupus erythematosus, cutaneous lupus erythematosus, and discoid lupus erythematosus, and cytokine release syndrome.

### CROSS-REFERENCE TO RELATED APPLICATIONS

This application is a continuation of U.S. application Ser. No. 18/737,724, filed Jun. 7, 2024, which is a continuation of U.S. application Ser. No. 16/095,475 filed Oct. 22, 2018, which was the U.S. National Stage of International Appl. No. PCT/US17/29802 filed Apr. 27, 2017, which claims priority to U.S. Provisional Appl. No. 62/328,959, filed Apr. 28, 2016, the content of which is incorporated by reference herein in their entirety.

### SEQUENCE LISTING

The present application contains a Sequence Listing, which has been submitted electronically through USPTO Patent Center in XML format and is hereby incorporated by reference in its entirety. Said XML copy, created on Jun. 9, 2025, is named “2011256-2502.xml” and is 40,585 bytes in size.

### FIELD OF THE INVENTION

The present application relates generally to pharmaceutical compositions and dosage regimens for the clinical use of anti-Blood Dendritic Cell Antigen 2 antibodies.

### BACKGROUND

Blood dendritic cell antigen 2 (BDCA2) is a C-type lectin expressed on human plasmacytoid dendritic cells (pDCs) (Dzionek et al., J. Immunol., 165:6037-6046 (2000)), a specialized population of bone marrow-derived cells that secrete type I interferons (IFNs) in response to toll-like receptor (TLR) ligands. BDCA2 consists of a single extracellular carbohydrate recognition domain (CRD), which belongs to the type II C-type lectin group, at its C-terminus, a transmembrane region, and a short cytoplasmic tail at its N-terminus that does not harbor a signaling motif. BDCA2 transmits intracellular signals through an associated transmembrane adaptor, the FcεRIγ, and induces a B cell receptor (BCR)-like signaling cascade.

### SUMMARY

This disclosure relates, in part, to compositions and dosage regimens of anti-BDCA2 antibodies or BDCA2-binding fragments thereof and their use in the treatment of BDCA2-associated disorders such as systematic lupus erythematosus (SLE), cutaneous lupus erythematosus (CLE), and discoid lupus erythematosus (DLE).

In one aspect, the disclosure features a pharmaceutical composition comprising an anti-BDCA2 antibody or BDCA2-binding fragment thereof, sucrose, and arginine hydrochloride (Arg·HCl).

In some embodiments, the anti-BDCA2 antibody or BDCA2-binding fragment thereof comprises an immunoglobulin heavy chain variable domain (VH) and an immunoglobulin light chain variable domain (VL), the VH and VL comprising the CDRs of BIIB059. In some instances, the six CDRs of BIIB059 comprise or consist of the amino acid sequences set forth in SEQ ID NO:1 or 17; SEQ ID NO:2; SEQ ID NO:3; SEQ ID NO:4; SEQ ID NO:5; and SEQ ID NO:6.

In some embodiments, the composition comprises the anti-BDCA2 antibody or BDCA2-binding fragment thereof at a concentration of 50 mg/ml to 225 mg/ml. In other embodiments, the composition comprises the anti-BDCA2 antibody or BDCA2-binding fragment thereof at a concentration of 125 mg/ml to 175 mg/ml. In certain embodiments, the composition comprises the anti-BDCA2 antibody or BDCA2-binding fragment thereof at a concentration of 150 mg/ml.

In some embodiments, the composition comprises sucrose at a concentration of 0.05% to 10%. In other embodiments, the composition comprises sucrose at a concentration of 1% to 5%.

In certain embodiments, the composition comprises sucrose at a concentration of 3%.

In some embodiments, the composition comprises Arg·HCl at a concentration of 50 mM to 250 mM. In other embodiments, the composition comprises Arg·HCl at a concentration of 75 mM to 125 mM. In certain embodiments, the composition comprises Arg·HCl at a concentration of 100 mM.

In some embodiments, the composition further comprises Polysorbate-80 (PS80). In some embodiments, the composition comprises PS80 at a concentration of 0.01% to 0.1%. In other embodiments, the composition comprises PS80 at a concentration of 0.03% to 0.08%. In certain embodiments, the composition comprises PS80 at a concentration of 0.05%.

In some embodiments, the composition further comprises histidine. In some embodiments, the composition comprises histidine at a concentration of 5 mM to 50 mM. In other embodiments, the composition comprises histidine at a concentration of 15 mM to 25 mM. In certain embodiments, the composition comprises histidine at a concentration of 20 mM.

In some embodiments, the composition has a pH of 5.3 to 5.7. In other embodiments, the composition has a pH of 5.5.

In some embodiments, the composition further comprises methionine. In some embodiments, the composition comprises methionine at a concentration of 1 mM to 20 mM. In other embodiments, the composition comprises methionine at a concentration of 5 mM to 15 mM. In certain embodiments, the composition comprises methionine at a concentration of 10 mM.

In some embodiments, the composition further comprises glutamic acid. In some embodiments, the composition comprises glutamic acid at a concentration of 50 mM to 100 mM. In other embodiments, the composition comprises glutamic acid at a concentration of 50 mM to 80 mM. In certain embodiments, the composition comprises glutamic acid at a concentration of 70 mM.

In some embodiments, the pharmaceutical composition comprises the anti-BDCA2 antibody or the BDCA2-binding fragment thereof at a concentration of 125 mg/ml to 175 mg/ml; sucrose at a concentration of 1% to 5%; histidine at a concentration of 15 mM to 25 mM; Arg·HCl at a concentration of 75 mM to 125 mM; and PS80 at a concentration of 0.03% to 0.08%. The composition has a pH of 5.3 to 5.7. In certain embodiments, the composition also comprises methionine at a concentration of 5 mM to 15 mM. In certain embodiments, the composition also comprises glutamic acid at a concentration of 60 mM to 80 mM.

In some embodiments, the pharmaceutical composition comprises the anti-BDCA2 antibody or the BDCA2-binding fragment thereof at a concentration of 150 mg/ml; sucrose at a concentration of 3%; histidine at a concentration of 20 mM; Arg·HCl at a concentration of 100 mM; and PS80 at a concentration of 0.05%. The composition has a pH of 5.5. In certain embodiments, the composition also comprises methionine at a concentration of 10 mM. In certain embodiments, the composition also comprises glutamic acid at a concentration of 70 mM.

In some embodiments, the VH comprises or consists of a sequence at least 80% identical to SEQ ID NO:7 and the VL comprises or consists of a sequence at least 80% identical to SEQ ID NO:8. In some embodiments, the VH comprises or consists of a sequence at least 90% identical to SEQ ID NO:7 and the VL comprises or consists of a sequence at least 90% identical to SEQ ID NO:8. In some embodiments, the VH comprises or consists of the sequence of SEQ ID NO:7 and the VL comprises or consists of the sequence of SEQ ID NO:8.

In some embodiments, the anti-BDCA2 antibody comprises an immunoglobulin heavy chain and an immunoglobulin light chain. In certain instances, the heavy chain comprises or consists of a sequence at least 80% identical to SEQ ID NO:9 and the light chain comprises or consists of a sequence at least 80% identical to SEQ ID NO:10. In other instances, the heavy chain comprises or consists of a sequence at least 90% identical to SEQ ID NO:9 and the light chain comprises or consists of a sequence at least 90% identical to SEQ ID NO:10. In yet other instances, the heavy chain comprises or consists of the sequence of SEQ ID NO:9 and the light chain comprises or consists of the sequence of SEQ ID NO:10.

In another aspect, the disclosure features a method of treating a condition selected from the group consisting of systemic lupus erythematosus, cutaneous lupus erythematosus, discoid lupus erythematosus, Sjogren's syndrome, dermatopolymyositis, scleroderma, and cytokine release syndrome in a human subject in need thereof. The method involves administering to the human subject a pharmaceutical composition described herein.

In some embodiments, the pharmaceutical composition is administered subcutaneously to the human subject.

In certain embodiments, the anti-BDCA2 antibody or BDCA2-binding fragment thereof of the pharmaceutical composition is administered to the human subject at a dose of 50 mg every four weeks.

In certain embodiments, the anti-BDCA2 antibody or BDCA2-binding fragment thereof of the pharmaceutical composition is administered to the human subject at a dose of 150 mg every four weeks.

In other embodiments, the anti-BDCA2 antibody or BDCA2-binding fragment thereof of the pharmaceutical composition is administered to the human subject at a dose of 450 mg every four weeks.

In another aspect, the disclosure provides a method of treating a condition selected from the group consisting of systemic lupus erythematosus, cutaneous lupus erythematosus, discoid lupus erythematosus, Sjogren's syndrome, dermatopolymyositis, scleroderma, and cytokine release syndrome in a human subject in need thereof. The method comprises administering subcutaneously to the human subject an anti-BDCA2 antibody or BDCA2-binding fragment thereof at a dose of 50 mg every four weeks. The anti-BDCA2 antibody or BDCA2-binding fragment thereof comprises an immunoglobulin heavy chain variable domain (VH) and an immunoglobulin light chain variable domain (VL). The VH and VL, respectively, comprise: VH complementarity determining regions (CDRs), wherein H-CDR1 consists of the amino acid sequence set forth in SEQ ID NO:1; H-CDR2 consists of the amino acid sequence set forth in SEQ ID NO:2; and H-CDR3 consists of the amino acid sequence set forth in SEQ ID NO:3; and VL CDRs, wherein L-CDR1 consists of the amino acid sequence set forth in SEQ ID NO:4; L-CDR2 consists of the amino acid sequence set forth in SEQ ID NO:5; and L-CDR3 consists of the amino acid sequence set forth in SEQ ID NO:6.

In some embodiments, the human subject is administered a loading dose of the anti-BDCA2 antibody or BDCA2-binding fragment thereof two weeks after the first administration of the anti-BDCA2 antibody or BDCA2-binding fragment thereof. In certain instances, the loading dose is 50 mg.

In another aspect, the disclosure provides a method of treating a condition selected from the group consisting of systemic lupus erythematosus, cutaneous lupus erythematosus, discoid lupus erythematosus, Sjogren's syndrome, dermatopolymyositis, scleroderma, and cytokine release syndrome in a human subject in need thereof. The method comprises administering subcutaneously to the human subject an anti-BDCA2 antibody or BDCA2-binding fragment thereof at a dose of 150 mg every four weeks. The anti-BDCA2 antibody or BDCA2-binding fragment thereof comprises an immunoglobulin heavy chain variable domain (VH) and an immunoglobulin light chain variable domain (VL). The VH and VL, respectively, comprise: VH complementarity determining regions (CDRs), wherein H-CDR1 consists of the amino acid sequence set forth in SEQ ID NO:1; H-CDR2 consists of the amino acid sequence set forth in SEQ ID NO:2; and H-CDR3 consists of the amino acid sequence set forth in SEQ ID NO:3; and VL CDRs, wherein L-CDR1 consists of the amino acid sequence set forth in SEQ ID NO:4; L-CDR2 consists of the amino acid sequence set forth in SEQ ID NO:5; and L-CDR3 consists of the amino acid sequence set forth in SEQ ID NO:6.

In some embodiments, the human subject is administered a loading dose of the anti-BDCA2 antibody or BDCA2-binding fragment thereof two weeks after the first administration of the anti-BDCA2 antibody or BDCA2-binding fragment thereof. In certain instances, the loading dose is 150 mg.

In another aspect, the disclosure provides a method of treating a condition selected from the group consisting of systemic lupus erythematosus, cutaneous lupus erythematosus, discoid lupus erythematosus, Sjogren's syndrome, dermatopolymyositis, scleroderma, and cytokine release syndrome in a human subject in need thereof. The method comprises administering subcutaneously to the human subject an anti-BDCA2 antibody or BDCA2-binding fragment thereof at a dose of 450 mg every four weeks. The anti-BDCA2 antibody or BDCA2-binding fragment thereof comprises an immunoglobulin heavy chain variable domain (VH) and an immunoglobulin light chain variable domain (VL). The VH and VL, respectively, comprise: VH complementarity determining regions (CDRs), wherein H-CDR1 consists of the amino acid sequence set forth in SEQ ID NO:1; H-CDR2 consists of the amino acid sequence set forth in SEQ ID NO:2; and H-CDR3 consists of the amino acid sequence set forth in SEQ ID NO:3; and VL CDRs, wherein L-CDR1 consists of the amino acid sequence set forth in SEQ ID NO:4; L CDR2 consists of the amino acid sequence set forth in SEQ ID NO:5; and L-CDR3 consists of the amino acid sequence set forth in SEQ ID NO:6.

In some embodiments, the human subject is administered a loading dose of the anti-BDCA2 antibody or BDCA2-binding fragment thereof two weeks after the first administration of the anti-BDCA2 antibody or BDCA2-binding fragment thereof. In certain instances, the loading dose is 450 mg.

These embodiments apply to all of the methods described above. In some embodiments, the human subject is administered at least 4 doses of the anti-BDCA2 antibody or antigen-binding fragment thereof. In some embodiments, the human subject is administered at least 7 doses of the anti-BDCA2 antibody or antigen-binding fragment thereof. In certain embodiments, the human subject is administered at least 10 doses of the anti-BDCA2 antibody or antigen-binding fragment thereof. In some embodiments, the VH comprises or consists of a sequence at least 80% identical to SEQ ID NO:7 and the VL comprises or consists of a sequence at least 80% identical to SEQ ID NO:8. In some embodiments, the VH comprises or consists of a sequence at least 90% identical to SEQ ID NO:7 and the VL comprises or consists of a sequence at least 90% identical to SEQ ID NO:8. In some embodiments, the VH comprises or consists of the sequence of SEQ ID NO:7 and the VL comprises or consists of the sequence of SEQ ID NO:8. In some embodiments, the anti-BDCA2 antibody comprises an immunoglobulin heavy chain and an immunoglobulin light chain. In certain instances, the heavy chain comprises or consists of a sequence at least 80% identical to SEQ ID NO:9 and the light chain comprises or consists of a sequence at least 80% identical to SEQ ID NO:10. In other instances, the heavy chain comprises or consists of a sequence at least 90% identical to SEQ ID NO:9 and the light chain comprises or consists of a sequence at least 90% identical to SEQ ID NO:10. In yet other instances, the heavy chain comprises or consists of the sequence of SEQ ID NO:9 and the light chain comprises or consists of the sequence of SEQ ID NO:10. In certain embodiments, the condition is systemic lupus erythematosus. In other embodiments, the condition is cutaneous lupus erythematosus (with or without SLE). In some embodiments, the condition is discoid lupus erythematosus (with or without SLE). In certain embodiments, the condition is cytokine release syndrome.

In another aspect, the disclosure features a syringe, injector (e.g., autoinjector, subcutaneous large volume injector), or pump comprising a sterile preparation of the pharmaceutical composition described herein adapted for subcutaneous administration of the anti-BDCA2 antibody or BDCA2-binding fragment thereof at a fixed dose of 50 mg, 150 mg, or 450 mg.

In another aspect, the disclosure provides a syringe, injector, or pump comprising a sterile preparation of an anti-BDCA2 antibody or BDCA2-binding fragment thereof. The syringe or pump is adapted for subcutaneous administration of the anti-BDCA2 antibody or BDCA2-binding fragment thereof at a fixed dose of 50 mg, 150 mg, or 450 mg. The anti-BDCA2 antibody or BDCA2-binding fragment thereof comprises an immunoglobulin heavy chain variable domain (VH) and an immunoglobulin light chain variable domain (VL). The VH and VL, respectively, comprise: VH complementarity determining regions (CDRs), wherein H-CDR1 consists of the amino acid sequence set forth in SEQ ID NO:1; H-CDR2 consists of the amino acid sequence set forth in SEQ ID NO:2; and H-CDR3 consists of the amino acid sequence set forth in SEQ ID NO:3; and VL CDRs, wherein L-CDR1 consists of the amino acid sequence set forth in SEQ ID NO:4; L CDR2 consists of the amino acid sequence set forth in SEQ ID NO:5; and L-CDR3 consists of the amino acid sequence set forth in SEQ ID NO:6.

In some embodiments, the VH comprises or consists of a sequence at least 80% identical to SEQ ID NO:7 and the VL comprises or consists of a sequence at least 80% identical to SEQ ID NO:8. In some embodiments, the VH comprises or consists of a sequence at least 90% identical to SEQ ID NO:7 and the VL comprises or consists of a sequence at least 90% identical to SEQ ID NO:8. In some embodiments, the VH comprises or consists of the sequence of SEQ ID NO:7 and the VL comprises or consists of the sequence of SEQ ID NO:8. In some embodiments, the anti-BDCA2 antibody comprises an immunoglobulin heavy chain and an immunoglobulin light chain. In certain instances, the heavy chain comprises or consists of a sequence at least 80% identical to SEQ ID NO:9 and the light chain comprises or consists of a sequence at least 80% identical to SEQ ID NO:10. In other instances, the heavy chain comprises or consists of a sequence at least 90% identical to SEQ ID NO:9 and the light chain comprises or consists of a sequence at least 90% identical to SEQ ID NO:10. In yet other instances, the heavy chain comprises or consists of the sequence of SEQ ID NO:9 and the light chain comprises or consists of the sequence of SEQ ID NO:10.

In another aspect, the disclosure provides a pharmaceutical composition comprising an anti-BDCA2 antibody or BDCA2-binding fragment thereof, sucrose, and arginine hydrochloride (Arg·HCl), wherein the pharmaceutical composition has a pH of 5.0 to 6.5. In certain embodiments of this aspect, sucrose is not part of the pharmaceutical composition.

In some embodiments, the anti-BDCA2 antibody or BDCA2-binding fragment thereof comprises an immunoglobulin heavy chain variable domain (VH) and an immunoglobulin light chain variable domain (VL), the VH and VL comprising the CDRs of BIIB059. In some instances, the six CDRs of BIIB059 comprise or consist of the amino acid sequences set forth in SEQ ID NO:1 or 17; SEQ ID NO:2; SEQ ID NO:3; SEQ ID NO:4; SEQ ID NO:5; and SEQ ID NO:6.

In some embodiments, the pharmaceutical composition comprises the anti-BDCA2 antibody or BDCA2-binding fragment thereof at a concentration of 50 mg/ml to 225 mg/ml. In some embodiments, the pharmaceutical composition comprises the anti-BDCA2 antibody or BDCA2-binding fragment thereof at a concentration of 125 mg/ml to 175 mg/ml. In other embodiments, the pharmaceutical composition comprises the anti-BDCA2 antibody or BDCA2-binding fragment thereof at a concentration of 150 mg/ml. In certain embodiments, the pharmaceutical composition comprises the anti-BDCA2 antibody or BDCA2-binding fragment thereof at a concentration of 200 mg/ml. In certain embodiments, the pharmaceutical composition comprises the anti-BDCA2 antibody or BDCA2-binding fragment thereof at a concentration of 225 mg/ml.

In some embodiments, the pharmaceutical composition comprises sucrose at a concentration of 1% to 10%. In some embodiments, the pharmaceutical composition comprises sucrose at a concentration of 1% to 5%. In certain embodiments, the pharmaceutical composition comprises sucrose at a concentration of 1%. In certain embodiments, the pharmaceutical composition comprises sucrose at a concentration of 3%.

In some embodiments, the composition comprises Arg·HCl at a concentration of 50 mM to 250 mM. In some embodiments, the composition comprises Arg·HCl at a concentration of 50 mM to 200 mM. In other embodiments, the composition comprises Arg·HCl at a concentration of 75 mM to 150 mM. In other embodiments, the composition comprises Arg·HCl at a concentration of 75 mM to 125 mM. In some embodiments, the composition comprises Arg·HCl at a concentration of 100 mM to 250 mM. In some embodiments, the composition comprises Arg·HCl at a concentration of 100 mM to 200 mM. In certain embodiments, the composition comprises Arg·HCl at a concentration of 100 mM. In certain embodiments, the composition comprises Arg·HCl at a concentration of 250 mM.

In some embodiments, the pharmaceutical composition comprises polysorbate-80. In certain instances, the composition comprises PS80 at a concentration of 0.02% to 0.08%. In other instances, the composition comprises PS80 at a concentration of 0.03% to 0.08%. In yet other instances, the composition comprises PS80 at a concentration of 0.05%.

In some embodiments, the pharmaceutical composition comprises histidine. In certain instances, the composition comprises histidine at a concentration of 10 mM to 30 mM. In other instances, the composition comprises histidine at a concentration of 15 mM to 25 mM. In yet other instances, the composition comprises histidine at a concentration of 20 mM.

In some embodiments, the pharmaceutical composition has a pH of 5.3 to 6.5. In certain instances, the composition has a pH of 5.3 to 6.0. In certain instances, the composition has a pH of 5.5. In certain instances, the composition has a pH of 6.0.

In some embodiments, the pharmaceutical composition comprises a thiol-containing antioxidant. In certain instances, the thiol-containing antioxidant is GSH, GSSG, the combination of GSH and GSSG, cystine, cysteine, or the combination of cysteine and cystine. In one instance, the thiol-containing antioxidant is GSH. In one instance, the thiol-containing antioxidant is GSSG. In yet another instance, the thiol-containing antioxidant is the combination of GSH and GSSG. In one instance, the thiol-containing antioxidant is cysteine. In yet another instance, the thiol-containing antioxidant is the combination of cysteine and cystine. In some instances, the thiol-containing antioxidant is found in the pharmaceutical composition at a concentration of 0.02 mM to 2 mM. In some instances, the thiol-containing antioxidant is found in the pharmaceutical composition at a concentration of 0.2 mM. In other instances, the thiol-containing antioxidant is found in the pharmaceutical composition at a concentration of 0.4 mM. In some instances, the thiol-containing antioxidant is found in the pharmaceutical composition at a concentration of 1.0 mM. In certain cases, GSH and GSSG are found in the pharmaceutical composition at concentrations of 0.4 mM and 0.2 mM, respectively. In other cases, cysteine and cystine are found in the pharmaceutical composition at concentrations of 0.4 mM and 0.2 mM, respectively.

In another aspect, the disclosure provides a pharmaceutical composition comprising an anti-Blood Dendritic Cell Antigen 2 (BDCA2) antibody or BDCA2-binding fragment thereof and histidine at a concentration of 10 mM to 30 mM, Arg·HCl at a concentration of 50 mM to 250 mM, and PS80 at a concentration of 0.02% to 0.08%, wherein the composition has a pH of 5.0 to 6.5.

In certain embodiments, the anti-BDCA2 antibody or BDCA2-binding fragment thereof comprises an immunoglobulin heavy chain variable domain (VH) and an immunoglobulin light chain variable domain (VL), the VH and VL, respectively, comprising VH complementarity determining regions (CDRs), wherein VH-CDR1 consists of the amino acid sequence set forth in SEQ ID NO:1 or 17; VH-CDR2 consists of the amino acid sequence set forth in SEQ ID NO:2; and VH-CDR3 consists of the amino acid sequence set forth in SEQ ID NO:3; and VL CDRs, wherein VL-CDR1 consists of the amino acid sequence set forth in SEQ ID NO:4; VL-CDR2 consists of the amino acid sequence set forth in SEQ ID NO:5; and VL-CDR3 consists of the amino acid sequence set forth in SEQ ID NO:6.

In certain embodiments, the pharmaceutical composition has an anti-BDCA2 antibody or the BDCA2-binding fragment thereof at a concentration of 50 mg/ml to 225 mg/ml.

In certain embodiments, the pharmaceutical composition comprises sucrose at a concentration of 1% to 10%.

In certain embodiments, the pharmaceutical composition comprises a thiol-containing antioxidant. In certain instances, the thiol-containing antioxidant is GSH, GSSG, the combination of GSH and GSSG, cystine, cysteine, or the combination of cysteine and cystine. In one instance, the thiol-containing antioxidant is GSH. In one instance, the thiol-containing antioxidant is GSSG. In yet another instance, the thiol-containing antioxidant is the combination of GSH and GSSG. In one instance, the thiol-containing antioxidant is cysteine. In yet another instance, the thiol-containing antioxidant is the combination of cysteine and cystine. In some instances, the thiol-containing antioxidant is found in the pharmaceutical composition at a concentration of 0.02 mM to 2 mM. In some instances, the thiol-containing antioxidant is found in the pharmaceutical composition at a concentration of 0.2 mM. In other instances, the thiol-containing antioxidant is found in the pharmaceutical composition at a concentration of 0.4 mM. In some instances, the thiol-containing antioxidant is found in the pharmaceutical composition at a concentration of 1.0 mM. In certain cases, GSH and GSSG are found in the pharmaceutical composition at concentrations of 0.4 mM and 0.2 mM, respectively. In other cases, cysteine and cystine are found in the pharmaceutical composition at concentrations of 0.4 mM and 0.2 mM, respectively.

In one embodiment, the pharmaceutical composition comprises the anti-BDCA2 antibody or the BDCA2-binding fragment thereof at a concentration of 150 mg/ml, sucrose at a concentration of 3%, histidine at a concentration of 20 mM, Arg·HCl at a concentration of 100 mM, PS80 at a concentration of 0.05%, and GSH or cysteine at a concentration of 0.4 mM. The composition has a pH of 5.5. In certain cases, the anti-BDCA2 antibody or BDCA2-binding fragment thereof comprises an immunoglobulin heavy chain variable domain (VH) and an immunoglobulin light chain variable domain (VL), the VH and VL, respectively, comprising VH complementarity determining regions (CDRs), wherein VH-CDR1 consists of the amino acid sequence set forth in SEQ ID NO:1 or 17; VH-CDR2 consists of the amino acid sequence set forth in SEQ ID NO:2; and VH-CDR3 consists of the amino acid sequence set forth in SEQ ID NO:3; and VL CDRs, wherein VL-CDR1 consists of the amino acid sequence set forth in SEQ ID NO:4; VL-CDR2 consists of the amino acid sequence set forth in SEQ ID NO:5; and VL-CDR3 consists of the amino acid sequence set forth in SEQ ID NO:6. In certain instances, sucrose is not part of this composition.

In another embodiment, the pharmaceutical composition comprises the anti-BDCA2 antibody or the BDCA2-binding fragment thereof at a concentration of 150 mg/ml, sucrose at a concentration of 3%, histidine at a concentration of 20 mM, Arg·HCl at a concentration of 100 mM, PS80 at a concentration of 0.05%, and GSSG or cystine at a concentration of 0.2 mM. The composition has a pH of 5.5. In certain cases, the anti-BDCA2 antibody or BDCA2-binding fragment thereof comprises an immunoglobulin heavy chain variable domain (VH) and an immunoglobulin light chain variable domain (VL), the VH and VL, respectively, comprising VH complementarity determining regions (CDRs), wherein VH-CDR1 consists of the amino acid sequence set forth in SEQ ID NO:1 or 17; VH-CDR2 consists of the amino acid sequence set forth in SEQ ID NO:2; and VH-CDR3 consists of the amino acid sequence set forth in SEQ ID NO:3; and VL CDRs, wherein VL-CDR1 consists of the amino acid sequence set forth in SEQ ID NO:4; VL-CDR2 consists of the amino acid sequence set forth in SEQ ID NO:5; and VL-CDR3 consists of the amino acid sequence set forth in SEQ ID NO:6. In certain instances, sucrose is not part of this composition.

In yet another embodiment, the pharmaceutical composition comprises the anti-BDCA2 antibody or the BDCA2-binding fragment thereof at a concentration of 150 mg/ml, sucrose at a concentration of 3%, histidine at a concentration of 20 mM, Arg·HCl at a concentration of 100 mM, PS80 at a concentration of 0.05%, and GSH (or cysteine) at a concentration of 0.4 mM and GSSG (or cystine) at a concentration of 0.2 mM. The composition has a pH of 5.5. In certain cases, the anti-BDCA2 antibody or BDCA2-binding fragment thereof comprises an immunoglobulin heavy chain variable domain (VH) and an immunoglobulin light chain variable domain (VL), the VH and VL, respectively, comprising VH complementarity determining regions (CDRs), wherein VH-CDR1 consists of the amino acid sequence set forth in SEQ ID NO:1 or 17; VH-CDR2 consists of the amino acid sequence set forth in SEQ ID NO:2; and VH-CDR3 consists of the amino acid sequence set forth in SEQ ID NO:3; and VL CDRs, wherein VL-CDR1 consists of the amino acid sequence set forth in SEQ ID NO:4; VL-CDR2 consists of the amino acid sequence set forth in SEQ ID NO:5; and VL-CDR3 consists of the amino acid sequence set forth in SEQ ID NO:6. In certain instances, sucrose is not part of this composition.

In another aspect, the disclosure features a pharmaceutical composition comprising an anti-BDCA2 antibody or the BDCA2-binding fragment thereof at a concentration of 200 mg/ml, sucrose at a concentration of 3%; histidine at a concentration of 20 mM, Arg·HCl at a concentration of 250 mM, and PS80 at a concentration of 0.05%. The composition has a pH of 6.0. This pharmaceutical composition is especially suitable for subcutaneous administration to a subject in need thereof. In certain cases, the anti-BDCA2 antibody or BDCA2-binding fragment thereof comprises an immunoglobulin heavy chain variable domain (VH) and an immunoglobulin light chain variable domain (VL), the VH and VL, respectively, comprising VH complementarity determining regions (CDRs), wherein VH-CDR1 consists of the amino acid sequence set forth in SEQ ID NO:1 or 17; VH-CDR2 consists of the amino acid sequence set forth in SEQ ID NO:2; and VH-CDR3 consists of the amino acid sequence set forth in SEQ ID NO:3; and VL CDRs, wherein VL-CDR1 consists of the amino acid sequence set forth in SEQ ID NO:4; VL-CDR2 consists of the amino acid sequence set forth in SEQ ID NO:5; and VL-CDR3 consists of the amino acid sequence set forth in SEQ ID NO:6. In certain instances, sucrose is not part of this composition.

In yet another aspect, the disclosure features a pharmaceutical composition comprising an anti-BDCA2 antibody or the BDCA2-binding fragment thereof at a concentration of 225 mg/ml, sucrose at a concentration of 1%; histidine at a concentration of 20 mM, Arg·HCl at a concentration of 250 mM, and PS80 at a concentration of 0.05%. The composition has a pH of 6.0. This pharmaceutical composition is especially suitable for subcutaneous administration to a subject in need thereof. In certain cases, the anti-BDCA2 antibody or BDCA2-binding fragment thereof comprises an immunoglobulin heavy chain variable domain (VH) and an immunoglobulin light chain variable domain (VL), the VH and VL, respectively, comprising VH complementarity determining regions (CDRs), wherein VH-CDR1 consists of the amino acid sequence set forth in SEQ ID NO:1 or 17; VH-CDR2 consists of the amino acid sequence set forth in SEQ ID NO:2; and VH-CDR3 consists of the amino acid sequence set forth in SEQ ID NO:3; and VL CDRs, wherein VL-CDR1 consists of the amino acid sequence set forth in SEQ ID NO:4; VL-CDR2 consists of the amino acid sequence set forth in SEQ ID NO:5; and VL-CDR3 consists of the amino acid sequence set forth in SEQ ID NO:6. In certain instances, sucrose is not part of this composition.

In certain embodiments of the above two aspects, the pharmaceutical composition comprises a thiol-containing antioxidant. In certain instances, the thiol-containing antioxidant is GSH, GSSG, the combination of GSH and GSSG, cystine, cysteine, or the combination of cysteine and cystine. In one instance, the thiol-containing antioxidant is GSH. In one instance, the thiol-containing antioxidant is GSSG. In yet another instance, the thiol-containing antioxidant is the combination of GSH and GSSG. In one instance, the thiol-containing antioxidant is cysteine. In yet another instance, the thiol-containing antioxidant is the combination of cysteine and cystine. In some instances, the thiol-containing antioxidant is found in the pharmaceutical composition at a concentration of 0.02 mM to 2 mM. In some instances, the thiol-containing antioxidant is found in the pharmaceutical composition at a concentration of 0.2 mM. In other instances, the thiol-containing antioxidant is found in the pharmaceutical composition at a concentration of 0.4 mM. In some instances, the thiol-containing antioxidant is found in the pharmaceutical composition at a concentration of 1.0 mM. In certain cases, GSH and GSSG are found in the pharmaceutical composition at concentrations of 0.4 mM and 0.2 mM, respectively. In other cases, cysteine and cystine are found in the pharmaceutical composition at concentrations of 0.4 mM and 0.2 mM, respectively.

These embodiments apply to all of the above aspects. In certain embodiments, the anti-BDCA2 antibody or BDCA2-binding fragment comprises a VH and VL, wherein the VH consists of a sequence at least 80% identical, at least 90% identical, at least 95% identical, at least 96% identical, at least 97% identical, at least 98% identical, at least 99% identical, or 100% identical to SEQ ID NO:7; and the VL consists of a sequence at least 80% identical, at least 90% identical, or at least 95% identical, at least 96% identical, at least 97% identical, at least 98% identical, at least 99% identical, or 100% identical to SEQ ID NO:8. In certain embodiments, the anti-BDCA2 antibody or BDCA2-binding fragment comprises an immunoglobulin heavy chain and an immunoglobulin light chain, wherein the heavy chain consists of a sequence at least 80% identical, at least 90% identical, at least 95% identical, at least 96% identical, at least 97% identical, at least 98% identical, at least 99% identical, or 100% identical to SEQ ID NO:9; and the light chain consists of a sequence at least 80% identical, at least 90% identical, at least 95% identical, at least 96% identical, at least 97% identical, at least 98% identical, at least 99% identical, or 100% identical to SEQ ID NO:10.

In another aspect, the disclosure features a method of treating a condition selected from the group consisting of systemic lupus erythematosus, cutaneous lupus erythematosus, discoid lupus erythematosus, Sjogren's syndrome, dermatopolymyositis, scleroderma, and cytokine release syndrome in a human subject in need thereof. The method comprises administering to the human subject a pharmaceutical composition comprising an anti-BDCA2 antibody or BDCA2-binding fragment described herein.

In certain embodiments, the pharmaceutical composition is administered subcutaneously to the human subject. In certain embodiments, the anti-BDCA2 antibody or BDCA2-binding fragment thereof of the pharmaceutical composition is administered to the human subject at a dose of 25 mg every four weeks. In certain embodiments, the anti-BDCA2 antibody or BDCA2-binding fragment thereof of the pharmaceutical composition is administered to the human subject at a dose of 50 mg every four weeks. In certain embodiments, the anti-BDCA2 antibody or BDCA2-binding fragment thereof of the pharmaceutical composition is administered to the human subject at a dose of 150 mg every four weeks. In certain embodiments, the anti-BDCA2 antibody or BDCA2-binding fragment thereof of the pharmaceutical composition is administered to the human subject at a dose of 450 mg every four weeks. In certain instances, the anti-BDCA2 antibody or BDCA2-binding fragment thereof of the pharmaceutical composition is administered to the human subject at the dose corresponding to the human subject's weight as recited below:

| Weight | | Dose |
|---|---|---|
| 10 to 18 | kg | 18 mg every four weeks |
| 18.1 to 25 | kg | 22 mg every four weeks |
| 25.1 to 48 | kg | 28 mg every four weeks |
| greater than 48 | kg | 50 mg every four weeks. |

In certain instances, the anti-BDCA2 antibody or BDCA2-binding fragment thereof of the pharmaceutical composition is administered to the human subject at the dose corresponding to the human subject's weight as recited below:

| Weight | | Dose | |
|---|---|---|---|
| 10 to 18 | kg | 40 | mg every four weeks |
| 18.1 to 25 | kg | 56 | mg every four weeks |
| 25.1 to 48 | kg | 80 | mg every four weeks |
| greater than 48 | kg | 150 | mg every four weeks. |

In another aspect, the disclosure provides a method of treating a condition selected from the group consisting of systemic lupus erythematosus, cutaneous lupus erythematosus, discoid lupus erythematosus, Sjogren's syndrome, dermatopolymyositis, scleroderma, and cytokine release syndrome in a human subject in need thereof. The method involves administering subcutaneously to the human subject an anti-BDCA2 antibody or BDCA2-binding fragment thereof at the dose corresponding to the human subject's weight as recited below:

| Weight | | Dose |
|---|---|---|
| 10 to 18 | kg | 18 mg every four weeks |
| 18.1 to 25 | kg | 22 mg every four weeks |
| 25.1 to 48 | kg | 28 mg every four weeks |
| greater than 48 | kg | 50 mg every four weeks. |

In certain cases, the anti-BDCA2 antibody or BDCA2-binding fragment thereof comprises an immunoglobulin heavy chain variable domain (VH) and an immunoglobulin light chain variable domain (VL), the VH and VL, respectively, comprising VH complementarity determining regions (CDRs), wherein VH-CDR1 consists of the amino acid sequence set forth in SEQ ID NO:1 or 17; VH-CDR2 consists of the amino acid sequence set forth in SEQ ID NO:2; and VH-CDR3 consists of the amino acid sequence set forth in SEQ ID NO:3; and VL CDRs, wherein VL-CDR1 consists of the amino acid sequence set forth in SEQ ID NO:4; VL-CDR2 consists of the amino acid sequence set forth in SEQ ID NO:5; and VL-CDR3 consists of the amino acid sequence set forth in SEQ ID NO:6. In certain embodiments, the anti-BDCA2 antibody or BDCA2-binding fragment comprises a VH and VL, wherein the VH consists of a sequence at least 80% identical, at least 90% identical, at least 95% identical, at least 96% identical, at least 97% identical, at least 98% identical, at least 99% identical, or 100% identical to SEQ ID NO:7; and the VL consists of a sequence at least 80% identical, at least 90% identical, or at least 95% identical, at least 96% identical, at least 97% identical, at least 98% identical, at least 99% identical, or 100% identical to SEQ ID NO:8. In certain embodiments, the anti-BDCA2 antibody or BDCA2-binding fragment comprises an immunoglobulin heavy chain and an immunoglobulin light chain, wherein the heavy chain consists of a sequence at least 80% identical, at least 90% identical, at least 95% identical, at least 96% identical, at least 97% identical, at least 98% identical, at least 99% identical, or 100% identical to SEQ ID NO:9; and the light chain consists of a sequence at least 80% identical, at least 90% identical, at least 95% identical, at least 96% identical, at least 97% identical, at least 98% identical, at least 99% identical, or 100% identical to SEQ ID NO:10. In certain embodiments, human subject is 20 years or less. In certain embodiments, human subject is 18 years or less. In certain embodiments, human subject is 16 years or less. In certain embodiments, human subject is 14 years or less. In certain embodiments, human subject is 12 years or less. In certain embodiments, human subject is 10 years or less. In certain embodiments, human subject is 8 years or less. In certain embodiments, human subject is 6 years or less. In certain embodiments, human subject is 4 years or less. In certain embodiments, human subject is 2 years or less.

In yet another aspect, the disclosure features a method of treating a condition selected from the group consisting of systemic lupus erythematosus, cutaneous lupus erythematosus, discoid lupus erythematosus, Sjogren's syndrome, dermatopolymyositis, scleroderma, and cytokine release syndrome in a human subject in need thereof. The method involves administering subcutaneously to the human subject an anti-BDCA2 antibody or BDCA2-binding fragment thereof at the dose corresponding to the human subject's weight as recited below:

| Weight | | Dose | |
|---|---|---|---|
| 10 to 18 | kg | 40 | mg every four weeks |
| 18.1 to 25 | kg | 56 | mg every four weeks |
| 25.1 to 48 | kg | 80 | mg every four weeks |
| greater than 48 | kg | 150 | mg every four weeks. |

In certain cases, the anti-BDCA2 antibody or BDCA2-binding fragment thereof comprises an immunoglobulin heavy chain variable domain (VH) and an immunoglobulin light chain variable domain (VL), the VH and VL, respectively, comprising VH complementarity determining regions (CDRs), wherein VH-CDR1 consists of the amino acid sequence set forth in SEQ ID NO:1 or 17; VH-CDR2 consists of the amino acid sequence set forth in SEQ ID NO:2; and VH-CDR3 consists of the amino acid sequence set forth in SEQ ID NO:3; and VL CDRs, wherein VL-CDR1 consists of the amino acid sequence set forth in SEQ ID NO:4; VL-CDR2 consists of the amino acid sequence set forth in SEQ ID NO:5; and VL-CDR3 consists of the amino acid sequence set forth in SEQ ID NO:6. In certain embodiments, the anti-BDCA2 antibody or BDCA2-binding fragment comprises a VH and VL, wherein the VH consists of a sequence at least 80% identical, at least 90% identical, at least 95% identical, at least 96% identical, at least 97% identical, at least 98% identical, at least 99% identical, or 100% identical to SEQ ID NO:7; and the VL consists of a sequence at least 80% identical, at least 90% identical, or at least 95% identical, at least 96% identical, at least 97% identical, at least 98% identical, at least 99% identical, or 100% identical to SEQ ID NO:8. In certain embodiments, the anti-BDCA2 antibody or BDCA2-binding fragment comprises an immunoglobulin heavy chain and an immunoglobulin light chain, wherein the heavy chain consists of a sequence at least 80% identical, at least 90% identical, at least 95% identical, at least 96% identical, at least 97% identical, at least 98% identical, at least 99% identical, or 100% identical to SEQ ID NO:9; and the light chain consists of a sequence at least 80% identical, at least 90% identical, at least 95% identical, at least 96% identical, at least 97% identical, at least 98% identical, at least 99% identical, or 100% identical to SEQ ID NO:10. In certain embodiments, human subject is 20 years or less. In certain embodiments, human subject is 18 years or less. In certain embodiments, human subject is 16 years or less. In certain embodiments, human subject is 14 years or less. In certain embodiments, human subject is 12 years or less. In certain embodiments, human subject is 10 years or less. In certain embodiments, human subject is 8 years or less. In certain embodiments, human subject is 6 years or less. In certain embodiments, human subject is 4 years or less. In certain embodiments, human subject is 2 years or less.

In another aspect, the disclosure features a syringe or pump comprising a sterile preparation of a pharmaceutical composition described herein adapted for subcutaneous administration of the anti-BDCA2 antibody or BDCA2-binding fragment thereof at a fixed dose of 18 mg, 22 mg, 25 mg, 28 mg, 40 mg, 50 mg, 56 mg, 80 mg, 150 mg, or 450 mg.

In another aspect, the disclosure features a syringe or pump comprising a sterile preparation of a pharmaceutical composition described herein adapted for subcutaneous administration of the anti-BDCA2 antibody or BDCA2-binding fragment thereof at a fixed dose of 18 mg, 22 mg, 25 mg, 28 mg, 40 mg, 50 mg, 56 mg, 80 mg, 150 mg, or 450 mg, wherein the anti-BDCA2 antibody or BDCA2-binding fragment thereof comprises an immunoglobulin heavy chain variable domain (VH) and an immunoglobulin light chain variable domain (VL), the VH and VL, respectively, comprising VH complementarity determining regions (CDRs), wherein VH-CDR1 consists of the amino acid sequence set forth in SEQ ID NO:1 or 17; VH-CDR2 consists of the amino acid sequence set forth in SEQ ID NO:2; and VH-CDR3 consists of the amino acid sequence set forth in SEQ ID NO:3; and VL CDRs, wherein VL-CDR1 consists of the amino acid sequence set forth in SEQ ID NO:4; VL-CDR2 consists of the amino acid sequence set forth in SEQ ID NO:5; and VL-CDR3 consists of the amino acid sequence set forth in SEQ ID NO:6. In certain embodiments, the anti-BDCA2 antibody or BDCA2-binding fragment comprises a VH and VL, wherein the VH consists of a sequence at least 80% identical, at least 90% identical, at least 95% identical, at least 96% identical, at least 97% identical, at least 98% identical, at least 99% identical, or 100% identical to SEQ ID NO:7; and the VL consists of a sequence at least 80% identical, at least 90% identical, or at least 95% identical, at least 96% identical, at least 97% identical, at least 98% identical, at least 99% identical, or 100% identical to SEQ ID NO:8. In certain embodiments, the anti-BDCA2 antibody or BDCA2-binding fragment comprises an immunoglobulin heavy chain and an immunoglobulin light chain, wherein the heavy chain consists of a sequence at least 80% identical, at least 90% identical, at least 95% identical, at least 96% identical, at least 97% identical, at least 98% identical, at least 99% identical, or 100% identical to SEQ ID NO:9; and the light chain consists of a sequence at least 80% identical, at least 90% identical, at least 95% identical, at least 96% identical, at least 97% identical, at least 98% identical, at least 99% identical, or 100% identical to SEQ ID NO:10.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1 is a graph depicting the viscosity of the antibody formulation.

FIG. 2A is a graph showing the aggregation of anti-BDCA2 antibodies formulated at a concentration of 150 mg/ml in a formulation containing 20 mM buffer as shown, 140 mM Arg·HCl, and 0.05% PS80 after 0-4 weeks of incubation at 40° C. Buffers are identified by the symbols shown in the figure.

FIG. 2B is a graph showing the aggregation of anti-BDCA2 antibodies formulated at a concentration of 150 mg/ml in a formulation containing 20 mM buffer as shown, 140 mM Arg·HCl, and 0.05% PS80 after 0-3 months of incubation at 5° C. Buffers are identified using the same symbols as shown in FIG. 2A.

FIG. 2C is a graph showing the aggregation of anti-BDCA2 antibodies formulated at a concentration of 200 mg/ml in a formulation containing 20 mM buffer as shown, 140 mM Arg·HCl, and 0.05% PS80 after 0-3 months of incubation at 5° C. Buffers are identified using the same symbols as shown in FIG. 2A.

FIG. 2D is a graph showing the aggregation of anti-BDCA2 antibodies formulated at a concentration of 225 mg/ml in a formulation containing 20 mM buffer as shown, 140 mM Arg·HCl, and 0.05% PS80 after 0-3 months of incubation at 5° C. Buffers are identified using the same symbols as shown in FIG. 2A.

FIG. 3 is a bar graph depicting the viscosity of anti-BDCA2 antibodies at different pH (5.5, 6, or 6.5), concentration (150 mg/ml, 225 mg/ml, or 250 mg/ml), and in different buffers (citrate or histidine).

FIG. 4 is a graph depicting aggregation of BDCA2 at 225 ng/ml in the formulations shown.

FIG. 5A is a bar graph showing sub-visible particulate formation (particles ≥2 μm) at time zero (first bar), after 2 weeks at 25° C. (second bar) or 2 weeks at 5° C. (third bar). Particle concentration is depicted on a log scale. Formulations contained the excipient(s) shown, as well as 20 mM Citrate pH 6.0, 0.05% PS80.

FIG. 5B is a bar graph showing sub-visible particulate formation (particles ≥10 μm) at time zero (first bar), after 2 weeks at 25° C. (second bar) or 2 weeks at 5° C. (third bar). Particle concentration is depicted on a log scale. Formulations contained the excipient(s) shown, as well as 20 mM Citrate, pH 6.0, and 0.05% PS80.

FIG. 6 is a bar graph depicting aggregation at time zero (first bar), after 2 weeks at 25° C. (second bar) or 2 weeks at 5° C. (third bar). Formulations contained the excipients shown as well as 20 mM Citrate, pH 6.0, and 0.05% PS80.

FIGS. 7A-7B provide graphs comparing aggregation of 150 mg/mL of anti-BDCA2 antibody formulated in Formulation 2 (20 mM His, 100 mM Arg·HCl, 3% sucrose, 0.05% PS80, pH 5.5) vs. Formulation 1 (20 mM Citrate, 140 mM Arg·HCl, 0.05% PS80, pH 6.0). FIG. 7A shows aggregation at 5° C. from 0 to 3 months; FIG. 7B shows aggregation at 25° C. from 0 to 3 months. Formulation 1 is indicated as “Cit 150” and Formulation 2 as “His 150” in the graphs.

FIG. 8 is a graph depicting the viscosity of anti-BDCA2 antibody in Formulation 2.

FIG. 9 is a graph showing the percentage of high molecular weight species that form over time at 5° C. in the ten formulations tested. The legend text corresponds to: protein concentration (mg/mL)/Arginine·HCl (mM)/Sucrose (%)/pH.

FIG. 10 is a graph showing the percentage of high molecular weight species that form over time at 25° C. in the ten formulations tested. The legend text corresponds to: protein concentration (mg/mL)/Arginine·HCl (mM)/Sucrose (%)/pH.

FIG. 11 is a graph showing the percentage of high molecular weight species that form over time at 30° C. in the ten formulations tested. The legend text corresponds to: protein concentration (mg/mL)/Arginine·HCl (mM)/Sucrose (%)/pH.

FIG. 12 is a graph showing the percentage of high molecular weight species that form over time at 40° C. in the ten formulations tested. The legend text corresponds to: protein concentration (mg/mL)/Arginine·HCl (mM)/Sucrose (%)/pH.

FIG. 13 is a graph showing the percentage of basic isoforms that form over time at 25° C. in the ten formulations tested. The legend text corresponds to: protein concentration (mg/mL)/Arginine·HCl (mM)/Sucrose (%)/pH.

FIG. 14 is a graph showing the percentage of basic isoforms that form over time at 30° C. in the ten formulations tested. The legend text corresponds to: protein concentration (mg/mL)/Arginine·HCl (mM)/Sucrose (%)/pH.

FIG. 15 is a graph showing the percentage of basic isoforms that form over time at 40° C. in the ten formulations tested. The legend text corresponds to: protein concentration (mg/mL)/Arginine·HCl (mM)/Sucrose (%)/pH.

FIG. 16 is a graph showing the percentage of basic isoforms that form over time at 5° C. in the ten formulations tested. The legend text corresponds to: protein concentration (mg/mL)/Arginine·HCl (mM)/Sucrose (%)/pH.

FIGS. 17A-17B provide graphs depicting the percentage of HMW species of an anti-BDCA2 antibody formulation comprising sucrose (150 mg/ml antibody; 20 mM histidine; 100 mM Arg·HCl; 3% sucrose; 0.05% PS80, pH 5.5) with or without GSH (0.4 mM) at 25° C. (FIG. 17A) and 40° C. (FIG. 17B).

FIGS. 18A-18B provide an overlay of the graph of FIG. 17 with a graph depicting the percentage of HMW species of an anti-BDCA2 antibody formulation lacking sucrose (150 mg/ml antibody; 20 mM histidine; 100 mM Arg·HCl; 0.05% PS80, pH 5.5) with or without GSH (0.4 mM) at 25° C. (FIG. 18A) and 40° C. (FIG. 18B). This shows that the presence of sucrose has no effect on GSH action.

FIGS. 19A-19B provide graphs depicting the percentage of HMW species of a BENEPALI® (an etanercept biosimilar referencing Enbrel®) formulation (50 mg/ml SB4; 10 mM sodium phosphate; 140 mM NaCl; 1% sucrose, pH 6.2) with or without GSH (0.4 mM) at 25° C. (FIG. 19A) and 40° C. (FIG. 19A).

FIGS. 20A-20B provide graphs depicting the percentage of HMW species of an anti-αvβ5 integrin antibody (STX200) formulation (50 mg/ml antibody; 20 mM histidine; 5% sorbitol; 0.05% PS80, pH 6.5) with or without GSH (0.4 mM) at 25° C. (FIG. 20A) and 40° C. (FIG. 20B).

### DETAILED DESCRIPTION

This application provides pharmaceutical compositions and dosage regimens of anti-BDCA2 antibodies and BDCA2-binding fragments thereof and their use in the treatment of BDCA2-associated disorders (e.g., SLE, CLE, and DLE).

### BDCA2

BDCA2 is a type II C-type lectin that is specifically expressed on plasmacytoid dendritic cells (pDCs). BDCA2 consists of a single extracellular carbohydrate recognition domain (CRD) at its C-terminus, a transmembrane region, and a short cytoplasmic tail at its N-terminus that does not harbor a signaling motif. BDCA2 transmits intracellular signals through an associated transmembrane adaptor, FcεRIγ. Antibody-mediated ligation of BDCA2 leads to recruitment of spleen tyrosine kinase (SYK) to phosphorylated immunoreceptor tyrosine-based activation motif (ITAM) of FcεRIγ. Syk activation leads to the activation of B cell linker (Blnk), Bruton's tyrosine kinase (BTK), and phospholipase Cy2 (PLCγ2), leading to Ca2⁺ mobilization.

The amino acid sequence of the human BDCA2 protein (Genbank® Accession No. NP_569708.1) is shown below (the transmembrane domain is italicized; the ectodomain is underlined).

| (SEQ ID NO: 29) | |
|---|---|
| 1 | MVPEEEPQDR EKGLWWFQLK VWSMAVSIL LLSVCFTVSS VVPHNFMYSK |
| 51 | TVKRLSKLRE YQQYHPSLTC VMEGKDIEDW SCCPTPWTSF QSSCYFISTG |
| 101 | MQSWTKSQKN CSVMGADLVV INTREEQDFI IQNLKRNSSY FLGLSDPGGR |
| 151 | RHWQWVDQTP YNENVTFWHS GEPNNLDERC AIINFRSSEE WGWNDIHCHV |
| 201 | PQKSICKMKK IYI* |

The amino acid sequence of the human FcεRIy (Genbank® Accession No. NP_004097.1) is shown below.

| (SEQ ID NO: 30) | |
|---|---|
| 1 | MIPAVVLLLL LLVEQAAALG EPQLCYILDA ILFLYGIVLT LLYCRLKIQV |
| 51 | RKAAITSYEK SDGVYTGLST RNQETYETLK HEKPPQ* |

### Anti-BDCA2 Antibodies

In some embodiments, the anti-BDCA2 antibody or BDCA2-binding fragment thereof used in the compositions and methods described herein comprises the three heavy chain variable domain complementarity determining regions (CDRs) of an antibody referred to as “BIIB059.” In some embodiments, the anti-BDCA2 antibody or BDCA2-binding fragment thereof comprises the three light chain variable domain CDRs of BIIB059. In still other embodiments, the anti-BDCA2 antibody or BDCA2-binding fragment thereof comprises the three heavy chain variable domain CDRs and the three light chain variable domain CDRs of BIIB059. The CDRs can be based on any CDR definition in the art, e.g., the definitions of Kabat, Chothia, Chothia from Abysis, enhanced Chothia/AbM, or based on the contact definition. CDR sequences of BIIB059 according to these exemplary CDR definitions are provided in Table 1 below.

TABLE 1
Sequences of the CDRs of BIIB059

| | | Chothia, | Enhanced | |
|---|---|---|---|---|
| Domain | Kabat | from Abysis | Chothia/AbM | Contact |
| VH CDR1 | TYTMS | GFTFSTY | GFTFSTYTMS | STYTMS |
| | (SEQ ID NO: 1) | (SEQ ID NO: 11) | (SEQ ID NO: 17) | (SEQ ID NO: 23) |
| VH CDR2 | TISPGDSFGYYYPDSVQG | SPGDSFG | TISPGDSFGYY | WVATISPGDSFGYY |
| | (SEQ ID NO: 2) | (SEQ ID NO: 12) | (SEQ ID NO: 18) | (SEQ ID NO: 24) |
| VH CDR3 | DIYYNYGAWFAY | DIYYNYGAWFAY | DIYYNYGAWFAY | TRDIYYNYGAWFA |
| | (SEQ ID NO: 3) | (SEQ ID NO: 13) | (SEQ ID NO: 19) | (SEQ ID NO: 25) |
| VL CDR1 | KASQSVDYDGDSYMN | KASQSVDYDGDSYMN | KASQSVDYDGDSYMN | DYDGDSYMNWY |
| | (SEQ ID NO: 4) | (SEQ ID NO: 14) | (SEQ ID NO: 20) | (SEQ ID NO: 26) |
| VL CDR2 | AASTLES | AASTLES | AASTLES | LLIYAASTLE |
| | (SEQ ID NO: 5) | (SEQ ID NO: 15) | (SEQ ID NO: 21) | (SEQ ID NO: 27) |
| VL CDR3 | QQANEDPRT | QQANEDPRT | QQANEDPRT | QQANEDPR |
| | (SEQ ID NO: 6) | (SEQ ID NO: 16) | (SEQ ID NO: 22) | (SEQ ID NO: 28) |

In some embodiments, the anti-BDCA2 antibody or BDCA2-binding fragment thereof comprises a VH CDR1 comprising or consisting of the amino acid sequence set forth in SEQ ID NO.:1 or 17, a VH CDR2 comprising or consisting of the amino acid sequence set forth in SEQ ID NO.: 2; and a VH CDR3 comprising or consisting of the amino acid sequence set forth in SEQ ID NO. 3. In some embodiments, the anti-BDCA2 antibody or BDCA2-binding fragment thereof comprises a VL CDR1 comprising or consisting of the amino acid sequence set forth in SEQ ID NO.:4, a VL CDR2 comprising or consisting of the amino acid sequence set forth in SEQ ID NO.: 5; and a VL CDR3 comprising or consisting of the amino acid sequence set forth in SEQ ID NO. 6.

In certain embodiments, the anti-BDCA2 antibody or BDCA2-binding fragment thereof comprises the CDRs comprising or consisting of the amino acid sequences set forth in SEQ ID NOs.: 1 to 6. In other embodiments, the anti-BDCA2 antibody or BDCA2-binding fragment thereof comprises the CDRs comprising or consisting of the amino acid sequences set forth in SEQ ID NOs.: 11 to 16. In yet other embodiments, the anti-BDCA2 antibody or BDCA2-binding fragment thereof comprises the CDRs comprising or consisting of the amino acid sequences set forth in SEQ ID NOs.: 17 to 22. In yet another embodiment, the anti-BDCA2 antibody or BDCA2-binding fragment thereof comprises the CDRs comprising or consisting of the amino acid sequences set forth in SEQ ID NOs.: 23 to 28. In one embodiment, the anti-BDCA2 antibody or BDCA2-binding fragment thereof comprises a VH CDR1 comprising or consisting of the amino acid sequence set forth in SEQ ID NO.:1 or 17, a VH CDR2 comprising or consisting of the amino acid sequence set forth in SEQ ID NO.: 2; and a VH CDR3 comprising or consisting of the amino acid sequence set forth in SEQ ID NO. 3; and a VL CDR1 comprising or consisting of the amino acid sequence set forth in SEQ ID NO:4, a VL CDR2 comprising or consisting of the amino acid sequence set forth in SEQ ID NO.: 5; and a VL CDR3 comprising or consisting of the amino acid sequence set forth in SEQ ID NO. 6.

BIIB059 is an exemplary anti-BDCA2 antibody that can be used in the compositions and methods described herein. BIIB059 is a humanized antibody having two glycosylated human IgG1 heavy chains and two human kappa light chains that specifically binds to BDCA2 on the surface of plasmacytoid dendritic cells. The wild-type IgG1 sequence contains a single N-linked glycosylation site and binds to Fc receptors with affinities typical of this class of molecules. This Fc function-competent IgG1 monoclonal antibody exhibits high affinity for BDCA2 and binds equally well to native human and cynomolgus BDCA2. BIIB059 is a potent inhibitor of all TLR9-induced type I interferons (IFNs) as well as other cytokines and chemokines by pDCs. BIIB059 is equally potent at inhibiting TLR9-induced type I interferon by pDCs from healthy human donors and SLE patients. BIIB059 specifically inhibits TLR9-induced type I IFN by pDCs and does not impact IFN production by other cell types triggered with different TLR ligand. BIIB059 also causes rapid internalization of BDCA2 from the cell surface. Upon stimulation, BDCA2 co-localizes with TLR9 in the endosomal/lysosomal compartment which appears to be necessary for its inhibition of TLR9 signaling. BIIB059 was also found to cause CD62L shedding from the surface of human pDCs. In vitro antibody-dependent cell-mediated cytotoxicity (ADCC) and complement-dependent cytotoxicity (CDC) studies suggest that BIIB059 can have cell depletion activity in cell lines overexpressing BDCA2.

The variable heavy chain (VH) of BIIB059 comprises or consists of the following amino acid sequence:

(SEQ ID NO: 7)

DVQLVESGGG LVKPGGSLRL SCAASGFTGS TYTMSWVRQA
PGKGLEWVAT ISPGDSFGYY YPDSVQGRFT ISRDNAKNSL
YLQMNSLRAE DTAVYYCTRD IYYNYGAWFA YWGQGTLVTV
SS

The variable light chain (VL) of BIIB059 comprises or consists of the following amino acid sequence:

(SEQ ID NO: 8)

DIQLTQSPSS LSASVGDRVT ITCKASQSVD YDGDSYMNWY
QQKPGKAPKL LIYAASTLES GVPSRFSGSG SGTDFTLTIS
SLQPEDFATY YCQQANEDPR TFGQGTKVEI K

In certain embodiments, the anti-BDCA2 antibody or BDCA2-binding fragment thereof comprises a VH having the amino acid sequence set forth in SEQ ID NO:7. In some embodiments, the anti-BDCA2 antibody or antigen-binding fragment thereof selectively binds to the ectodomain of human BDCA2 and comprises a VH domain that is at least 70%, 75%, 80%, 85%, 90%, 91%, 92%, 93%, 94%, 95%, 96%, 97%, 98%, 99% or more identical to the amino acid sequence of the VH domain of BIIB059 (SEQ ID NO:7), or differs at least at 1 to 5 amino acid residues, but at fewer than 40, 30, 20, 15, or 10, residues, from SEQ ID NO:7. In certain instances, these antibodies (i) bind human or cynomolgus monkey BDCA2 but do not significantly bind BDCA2 from phylogenetic species below primates; and/or (ii) inhibit TLR7/TLR9-induced type I interferon and other cytokine or chemokine production by human pDCs; and/or (iii) mediate internalization of BDCA2 from the surface of pDCs; and/or (iv) downregulate CD32a and/or CD62L from the surface of pDCs; and/or (v) deplete pDCs in vitro by ADCC or CDC.

In certain embodiments, the anti-BDCA2 antibody or BDCA2-binding fragment thereof comprises a VL having the amino acid sequence set forth in SEQ ID NO:8. In some embodiments, the anti-BDCA2 antibody or antigen-binding fragment thereof selectively binds to the ectodomain of human BDCA2 and comprises a VL domain that is at least 70%, 75%, 80%, 85%, 90%, 91%, 92%, 93%, 94%, 95%, 96%, 97%, 98%, 99% or more identical to the amino acid sequence of the VL domain of BIIB059 (SEQ ID NO:8), or differs at least at 1 to 5 amino acid residues, but at fewer than 40, 30, 20, 15, or 10, residues, from SEQ ID NO:8. In certain instances, these antibodies (i) bind human or cynomolgus monkey BDCA2 but do not significantly bind BDCA2 from phylogenetic species below primates; and/or (ii) inhibit TLR7/TLR9-induced type I interferon and other cytokine or chemokine production by human pDCs; and/or (iii) mediate internalization of BDCA2 from the surface of pDCs; and/or (iv) downregulate CD32a and/or CD62L from the surface of pDCs; and/or (v) deplete pDCs in vitro by ADCC or CDC.

In some embodiments, the anti-BDCA2 antibody or BDCA2-binding fragment thereof comprises a VH having the amino acid sequence set forth in SEQ ID NO:7 and a VL having the amino acid sequence set forth in SEQ ID NO:8. In some embodiments, the anti-BDCA2 antibody or antigen-binding fragment thereof selectively binds to the ectodomain of human BDCA2 and comprises (i) a VH domain that is at least 70%, 75%, 80%, 85%, 90%, 91%, 92%, 93%, 94%, 95%, 96%, 97%, 98%, 99% or more identical to the amino acid sequence of the VH domain of BIIB059 (SEQ ID NO:7), and (ii) a VL domain that is at least 70%, 75%, 80%, 85%, 90%, 91%, 92%, 93%, 94%, 95%, 96%, 97%, 98%, 99% or more identical to the amino acid sequence of the VL domain of BIIB059 (SEQ ID NO:8); or differs at least at 1 to 5 amino acid residues, but at fewer than 40, 30, 20, 15, or 10, residues, from SEQ ID NO:7 and/or SEQ ID NO:8. In certain instances, these antibodies (i) bind human or cynomolgus monkey BDCA2 but do not significantly bind BDCA2 from phylogenetic species below primates; and/or (ii) inhibit TLR7/TLR9-induced type I interferon and other cytokine or chemokine production by human pDCs; and/or (iii) mediate internalization of BDCA2 from the surface of pDCs; and/or (iv) downregulate CD32a and/or CD62L from the surface of pDCs; and/or (v) deplete pDCs in vitro by ADCC or CDC.

An antibody consisting of the mature heavy chain (SEQ ID NO:9) and the mature light chain (SEQ ID NO:10) listed below is termed “BIIB059” as used herein.

Mature BIIB059 heavy chain (HC)

(SEQ ID NO: 9)

DVQLVESGGG LVKPGGSLRL SCAASGFTGS TYTMSWVRQA
PGKGLEWVAT ISPGDSFGYY YPDSVQGRFT ISRDNAKNSL
YLQMNSLRAE DTAVYYCTRD IYYNYGAWFA YWGQGTLVTV
SSASTKGPSV FPLAPSSKST SGGTAALGCL VKDYFPEPVT
VSWNSGALTS GVHTFPAVLQ SSGLYSLSSV VTVPSSSLGT
QTYICNVNHK PSNTKVDKKV EPKSCDKTHT CPPCPAPELL
GGPSVFLFPP KPKDTLMISR TPEVTCVVVD VSHEDPEVKF
NWYVDGVEVH NAKTKPREEQ YNSTYRVVSV LTVLHQDWLN
GKEYKCKVSN KALPAPIEKT ISKAKGQPRE PQVYTLPPSR
DELTKNQVSL TCLVKGFYPS DIAVEWESNG QPENNYKTTP
PVLDSDGSFF LYSKLTVDKS RWQQGNVFSC SVMHEALHNH
YTQKSLSLSP G
Mature BIIB059 light chain (LC)

(SEQ ID NO: 10)

DIQLTQSPSS LSASVGDRVT ITCKASQSVD YDGDSYMNWY
QQKPGKAPKL LIYAASTLES GVPSRFSGSG SGTDFTLTIS
SLQPEDFATY YCQQANEDPR TFGQGTKVEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVT KSFNRGEC

In the above VH, VL, HC, and LC sequences, CDRs 1, 2, and 3 based on the Kabat definition are both underlined and boldened. The italicized and boldened sequence in the VH and HC is the additional N-terminal sequence found in the CDR1 based on enhanced Chothia/AbM definition.

In certain embodiments, the anti-BDCA2 antibody or BDCA2-binding fragment thereof comprises a HC having the amino acid sequence set forth in SEQ ID NO:9. In some embodiments, the anti-BDCA2 antibody or antigen-binding fragment thereof selectively binds to the ectodomain of human BDCA2 and comprises a HC that is at least 70%, 75%, 80%, 85%, 90%, 91%, 92%, 93%, 94%, 95%, 96%, 97%, 98%, 99% or more identical to the amino acid sequence of SEQ ID NO:9, or differs at least at 1 to 5 amino acid residues, but at fewer than 40, 30, 20, 15, or 10, residues, from SEQ ID NO:9.

In certain embodiments, the anti-BDCA2 antibody or BDCA2-binding fragment thereof comprises a LC having the amino acid sequence set forth in SEQ ID NO:10. In some embodiments, the anti-BDCA2 antibody or antigen-binding fragment thereof selectively binds to the ectodomain of human BDCA2 and comprises a LC that is at least 70%, 75%, 80%, 85%, 90%, 91%, 92%, 93%, 94%, 95%, 96%, 97%, 98%, 99% or more identical to the amino acid sequence of SEQ ID NO:10, or differs at least at 1 to 5 amino acid residues, but at fewer than 40, 30, 20, 15, or 10, residues, from SEQ ID NO:10.

In some embodiments, the anti-BDCA2 antibody or BDCA2-binding fragment thereof comprises a HC having the amino acid sequence set forth in SEQ ID NO:9 and a LC having the amino acid sequence set forth in SEQ ID NO:10. In some embodiments, the anti-BDCA2 antibody or antigen-binding fragment thereof selectively binds to the ectodomain of human BDCA2 and comprises (i) a HC that is at least 70%, 75%, 80%, 85%, 90%, 91%, 92%, 93%, 94%, 95%, 96%, 97%, 98%, 99% or more identical to the amino acid sequence of SEQ ID NO:9, and (ii) a LC that is at least 70%, 75%, 80%, 85%, 90%, 91%, 92%, 93%, 94%, 95%, 96%, 97%, 98%, 99% or more identical to the amino acid sequence of SEQ ID NO:10; or differs at least at 1 to 5 amino acid residues, but at fewer than 40, 30, 20, 15, or 10, residues, from SEQ ID NO:9 and/or SEQ ID NO:10.

In certain embodiments, the anti-BDCA2 antibody is an IgG antibody. In specific embodiments, the anti-BDCA2 antibody has heavy chain constant region chosen from, e.g., IgG1, IgG2, IgG3, IgG4, IgM, IgA1, IgA2, IgD, and IgE. In one embodiment, the anti-BDCA2 antibody is of the IgG1 isotype. In another embodiment, the anti-BDCA2 antibody is of the IgG2 isotype. In yet another embodiment, the anti-BDCA2 antibody is of the IgG3 isotype. In further embodiments, the antibody has a light chain constant region chosen from, e.g., a human kappa or human lambda light chain. In a certain embodiment, the anti-BDCA2 antibody is an IgG1/kappa antibody. In certain embodiments, the anti-BDCA2 antibody includes a human Fc region that binds FcγRIIa (CD32a) with an EC₅₀ of 7 to 15 g/mL. In certain embodiments, the antibody includes a human Fc region that binds FcγRIIa (CD32a) with an EC₅₀ of 10 g/mL. In certain embodiments, the antibody includes a human Fc region that binds FcγRIIa (CD32a) with an EC₅₀ of 11 g/mL. In certain embodiments, the antibody includes a human Fc region that binds FcγRIIa (CD32a) with an EC₅₀ of 12 g/mL. In some cases, the heavy chain constant region is human or a modified form of a human constant region. In certain instances the human constant region may include at least 1 and up to 2, 3, 4, 5, 6, 7, 8, 9, 10, 11, 12, 13, 14, 15, 16, 17, 18, 19, or 20 substitutions. In a particular embodiment, the modified human Fc region is a modified human IgG1 Fc region. In some cases, the constant region of an anti-BDCA2 antibody may be modified by mutation of one or more amino acid residues to impart a desired functional property (e.g., altered effector function or half-life, reduced glycosylation). For example, the N-linked glycosylation site may be substituted to prevent or reduce N-linked glycosylation of Fc region (e.g., human IgG1 Fc region).

In some embodiments, the anti-BDCA2 antibody is a full-length (whole) antibody or substantially full-length. The protein can include at least one, and preferably two, complete heavy chains, and at least one, and preferably two, complete light chains. In some embodiments, the anti-BDCA2 antibody is a BDCA2-binding fragment. In some instances, the BDCA2-binding fragment is a Fab, a Fab′, an F(ab′)₂, a Facb, an Fv, a single chain Fv (scFv), a sc(Fv)2, or a diabody.

Antibodies, such as BIIB059, or BDCA2-binding fragments thereof can be made, for example, by preparing and expressing synthetic genes that encode the recited amino acid sequences or by mutating human germline genes to provide a gene that encodes the recited amino acid sequences. Moreover, this antibody and other anti-BDCA2 antibodies can be produced, e.g., using one or more of the following methods.

### Methods of Producing Antibodies

Anti-BDCA2 antibodies or BDCA2-binding fragments may be produced in bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hanseula, or Saccharomyces. To produce the antibody of interest, a polynucleotide encoding the antibody is constructed, introduced into an expression vector, and then expressed in suitable host cells. Polynucleotides encoding an anti-BDCA2 antibody comprising the VH and/or VL, HC and/or LC of the BDCA2 antibodies described herein would be readily envisioned by the ordinarily skilled artisan. Standard molecular biology techniques are used to prepare the recombinant expression vector, transfect the host cells, select for transformants, culture the host cells and recover the antibody.

If the anti-BDCA2 antibodies or BDCA2-binding fragments is to be expressed in bacterial cells (e.g., E. coli), the expression vector should have characteristics that permit amplification of the vector in the bacterial cells. Additionally, when E. coli such as JM109, DH55a, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-BDCA2 antibody (e.g., BIIB059) is introduced into dhfr CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Anti-BDCA2 Antibody Compositions

This disclosure also provides compositions (e.g., pharmaceutical compositions) comprising the anti-BDCA2 antibodies or BDCA2-binding fragments thereof described herein. For example, the anti-BDCA2 antibody compositions comprises an anti-BDCA2 antibody or BDCA2-binding fragment thereof comprising an immunoglobulin heavy chain variable domain (VH) and an immunoglobulin light chain variable domain (VL), wherein the VH comprises the H-CDRs and the VL comprises the L-CDRs of BIIB059. In certain instances, the H-CDRs of comprise or consist of the amino acid sequences set forth in SEQ ID NO:1 or 17, SEQ ID NO:2, and SEQ ID NO:3; and the L-CDRs comprise or consist of the amino acid sequences set forth in SEQ ID NO:4, SEQ ID NO:5, and SEQ ID NO:6. In some embodiments, the anti-BDCA2 antibody compositions comprises an anti-BDCA2 antibody or BDCA2-binding fragment thereof comprising (i) a VH comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:7; and (ii) a VL comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:8. In certain embodiments, the anti-BDCA2 antibody compositions comprises an anti-BDCA2 antibody comprising (i) a heavy chain comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:9; and (ii) a light chain comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:10.

In certain embodiments, these compositions are high concentration anti-BDCA2 antibody composition. By “high concentration anti-BDCA2 antibody composition” is meant a composition comprising anti-BDCA2 antibodies or BDCA2-binding fragments thereof at a concentration of greater than 50 mg/ml and less than 300 mg/ml. In certain instances, the anti-BDCA2 antibody composition comprises anti-BDCA2 antibodies or BDCA2-binding fragments thereof at a concentration of 50 mg/ml to 240 mg/ml. In certain instances, the anti-BDCA2 antibody composition comprises anti-BDCA2 antibodies or BDCA2-binding fragments thereof at a concentration of 50 mg/ml to 225 mg/ml. In other instances, the anti-BDCA2 antibody composition comprises anti-BDCA2 antibodies or BDCA2-binding fragments thereof at a concentration of 75 mg/ml to 225 mg/ml. In other instances, the anti-BDCA2 antibody composition comprises anti-BDCA2 antibodies or BDCA2-binding fragments thereof at a concentration of 100 mg/ml to 225 mg/ml. In yet other instances, the anti-BDCA2 antibody composition comprises anti-BDCA2 antibodies or BDCA2-binding fragments thereof at a concentration of 125 mg/ml to 225 mg/ml. In other instances, the anti-BDCA2 antibody composition comprises anti-BDCA2 antibodies or BDCA2-binding fragments thereof at a concentration of 125 mg/ml to 175 mg/ml. In certain instances, the anti-BDCA2 antibody composition comprises anti-BDCA2 antibodies or BDCA2-binding fragments thereof at a concentration of 240 mg/ml. In certain instances, the anti-BDCA2 antibody composition comprises anti-BDCA2 antibodies or BDCA2-binding fragments thereof at a concentration of 225 mg/ml. In certain instances, the anti-BDCA2 antibody composition comprises anti-BDCA2 antibodies or BDCA2-binding fragments thereof at a concentration of 200 mg/ml. In certain instances, the anti-BDCA2 antibody composition comprises anti-BDCA2 antibodies or BDCA2-binding fragments thereof at a concentration of 175 mg/ml. In certain instances, the anti-BDCA2 antibody composition comprises anti-BDCA2 antibodies or BDCA2-binding fragments thereof at a concentration of 150 mg/ml. In other instances, the anti-BDCA2 antibody composition comprises anti-BDCA2 antibodies or BDCA2-binding fragments thereof at a concentration of 125 mg/ml. In some instances, the anti-BDCA2 antibody composition comprises anti-BDCA2 antibodies or BDCA2-binding fragments thereof at a concentration of 100 mg/ml.

A composition (e.g., a pharmaceutical composition) comprising an anti-BDCA2 antibody or BDCA2-binding fragment thereof described herein may be in any one of a variety of forms. These include, for example, liquid solutions (e.g., injectable and infusible solutions), dispersions, or suspensions. The preferred form can depend on the intended mode of administration and therapeutic application. In certain embodiments, a pharmaceutical composition described herein is in the form of a sterile injectable or infusible solution.

Sterile injectable solutions can be prepared by incorporating an antibody described herein in the required amount with one or a combination of ingredients, followed by filtered sterilization. Generally, dispersions are prepared by incorporating an antibody described herein into a sterile vehicle that contains a basic dispersion medium and the required other ingredients. In the case of sterile powders for the preparation of sterile injectable solutions, an exemplary method of preparation is vacuum drying and freeze drying that yields a powder of an antibody described herein plus any additional desired ingredient from a previously sterile-filtered solution thereof. The proper fluidity of a solution can be maintained, for example, by the use of a coating such as lecithin, by the maintenance of the required particle size in the case of dispersion, and by the use of surfactants.

The anti-BDCA2 antibody compositions (e.g., pharmaceutical compositions) may additionally comprise one or more excipients.

In one embodiment, the excipient lowers/reduces the aggregation and/or viscosity of the antibody in the composition compared to aggregation and/or viscosity of the antibody in the pharmaceutical composition without that excipient. In certain embodiments, such an excipient is arginine. In one instance, the excipient is arginine hydrochloride. Arginine (e.g., arginine hydrochloride) can be included in the composition at a concentration of 50 mM to 250 mM, 50 mM to 200 mM, 50 mM to 150 mM, 50 mM to 125 mM, 50 mM to 100 mM, 75 mM to 250 mM, 75 mM to 200 mM, 75 mM to 150 mM, or 75 mM to 100 mM. In certain embodiments arginine (e.g., Arg·HCl) is present in the composition at a concentration of 50 mM to 250 mM. In other embodiments, arginine (e.g., Arg·HCl) is present in the composition at a concentration of 50 mM to 200 mM. In certain instances, arginine (e.g., arginine hydrochloride) can be included in the composition at a concentration of 100 mM, 120 mM, 125 mM, 130 mM, 135 mM, 140 mM, 145 mM, or 150 mM. In a specific instance, arginine (e.g., arginine hydrochloride) can be included in the composition at a concentration of 100 mM. In another specific instance, arginine (e.g., arginine hydrochloride) can be included in the composition at a concentration of 250 mM.

Sometimes, solutions containing arginine develop visible particles after incubation at room temperature or higher temperatures (e.g., 40° C.). Surprisingly, it was found that addition of sucrose can reduce or prevent the formation of visible particles. Furthermore, sucrose was also unexpectedly found to lower the counts of subvisible particulates. In some embodiments, the anti-BDCA2 antibody composition comprises sucrose at a concentration of 0.05% to 15%, 0.05% to 10%, 0.05% to 5%, 1% to 15%, 1% to 10%, 1% to 5%, 2% to 8%, 2% to 6%, or 2% to 4%. In certain embodiments, the anti-BDCA2 antibody composition comprises sucrose at a concentration of 0.5%, 1%, 1.5%, 2%, 2.5%, 3%, 3.5%, 4%, 4.5% 5%, 5.5%, 6%, 6.5%, 7%, 7.5%, 8%, 8.5%, 9%, 9.5% or 10%. In a particular embodiment, the anti-BDCA2 antibody composition comprises sucrose at a concentration of 3%. In another particular embodiment, the anti-BDCA2 antibody composition comprises sucrose at a concentration of 1%.

Antibody product manufacturing is a complex process that can involve several steps such as, e.g., drug substance and bulk formulation, filtration, shipping, pooling, filling, lyophilization, inspections, packaging, and storage. During these steps, antibodies may be subjected to many different forms of stresses, e.g., agitation, temperature, light exposure, and oxidation. These types of stresses can lead to denaturation and aggregation of the antibody, which compromise the product quality and can even lead to loss of a production batch. Agitation is one of the common physical stresses that antibody therapeutics are subjected to during the course of the manufacturing process. Agitation occurs, e.g., during mixing, ultrafiltration/diafiltration, pumping, shipping, and filling. To protect the antibody composition against agitation-induced stress, the composition may include a polysorbate. In certain embodiments, the composition comprises polysorbate-80 at a concentration of 0.01% to 0.5%, 0.01% to 0.1%, 0.01% to 0.09%, 0.01% to 0.08%, 0.01% to 0.07%, 0.01% to 0.06%, 0.01% to 0.05%, 0.01% to 0.04%, or 0.01% to 0.03%. In certain embodiments, the composition comprises polysorbate-80 at a concentration of 0.02% to 0.08%. In some embodiments, the composition comprises polysorbate-80 at a concentration of 0.01%, 0.02%, 0.03%, 0.04%, 0.05%, 0.06%, 0.07%, 0.08%, 0.09%, or 0.1%. In a particular embodiment, the composition comprises polysorbate-80 at a concentration of 0.05%.

Any antibody composition benefits from a buffer that provides good buffering capacity. In certain embodiments, the antibody composition comprises histidine as the buffering agent. In certain embodiments, the composition comprises histidine at a concentration of 5 mM to 50 mM, 5 mM to 40 mM, 5 mM to 30 mM, 5 mM to 25 mM, 10 mM to 50 mM, 10 mM to 40 mM, 10 mM to 30 mM, 10 mM to 25 mM, 15 mM to 50 mM, 15 mM to 40 mM, 15 mM to 30 mM, or 15 mM to 25 mM. In certain embodiments, the composition comprises histidine at a concentration of 10 mM to 30 mM. In some embodiments, the composition comprises histidine at a concentration of 5 mM, 10 mM, 15 mM, 20 mM, 25 mM, or 30 mM. In a particular embodiment, the composition comprises histidine at a concentration of 20 mM.

The pH of the antibody composition can be 5.0 to 6.5. In certain cases, the pH of the antibody composition can be 5.0 to 6.0. In certain instances, the pH of the antibody composition is 5.0, 5.1, 5.2, 5.3, 5.4, 5.5, 5.6, 5.7, 5.8, 5.9, 6.0, 6.1, 6.2, 6.3, 6.4, or 6.5. In a particular embodiment, the pH of the antibody composition is 5.5. In another particular embodiment, the pH of the antibody composition is 6.0. In yet another particular embodiment, the pH of the antibody composition is 6.5.

In certain embodiments, the composition comprises a thiol-containing antioxidant (e.g., reduced glutathione (GSH), oxidized glutathione (GSSG), GSH+GSSG, cysteine, cystine, cysteine+cystine) at a concentration of 0.02 mM to 2 mM (e.g., 0.02, 0.03, 0.05, 0.06, 0.08, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mM). Such thiol-containing antioxidants can cleave unfavorable or misbridged disulfide bonds and promote the formation of favorable or properly bridged disulfide bonds. This would result in the stabilization of the native confirmation of the antibody or fragment thereof and slow down aggregation rates. The antioxidant properties of these molecules may slow down oxidative processes that lead to aggregation. In some cases, the composition comprises GSH at a concentration of 0.4 mM. In some cases, the composition comprises GSSG at a concentration of 0.2 mM. In some cases, the composition comprises GSH at a concentration of 0.4 mM and GSSG at a concentration of 0.2 mM. In some cases, the composition comprises cysteine at a concentration of 0.4 mM. In some cases, the composition comprises cystine at a concentration of 0.2 mM. In some cases, the composition comprises cysteine at a concentration of 0.4 mM and cystine at a concentration of 0.2 mM. In certain embodiments, the composition comprises methionine at a concentration of 5 mM to 15 mM (e.g., 10 mM). In certain embodiments, the composition comprises glutamic acid at a concentration of 50 mM to 80 mM (e.g., 70 mM).

In certain embodiments, the composition (e.g., a pharmaceutical composition) comprises an anti-BDCA2 antibody or a BDCA2-binding fragment thereof at a concentration of 50 mg/ml to 225 mg/ml, sucrose at a concentration of 0.05% to 10%, arginine (e.g., arginine hydrochloride) at a concentration of 50 mM to 250 mM, polysorbate-80 at a concentration of 0.01% to 0.1%, and histidine at a concentration of 10 mM to 30 mM. The composition has a pH of 5.0 to 6.0. In certain embodiments, the anti-BDCA2 antibody or BDCA2-binding fragment thereof of the composition comprises a VH and a VL comprising the CDRs of BIIB059 (e.g., SEQ ID NOs.: 1 or 17, 2, 3, 4, 5, and 6). In certain embodiments, the anti-BDCA2 antibody or BDCA2-binding fragment thereof of the composition comprises a VH and a VL comprising SEQ ID NOs: 7 and 8, respectively. In some embodiments, the anti-BDCA2 antibody or BDCA2-binding fragment thereof of the composition comprises a heavy chain and a light chain comprising SEQ ID NOs: 9 and 10, respectively. In one embodiment, the composition has a pH of 5.5 and comprises BIIB059 or a BIIB059-binding fragment thereof at a concentration of 150 mg/ml, sucrose at a concentration of 3%, arginine hydrochloride at a concentration of 100 mM, polysorbate-80 at a concentration of 0.05%, and histidine at a concentration of 20 mM. This embodiment can be made by, e.g., dissolving in 1833.50 mg sterile water (e.g., reverse osmosis deionized water (RODI)), 285 mg of BIIB059, 6.69 mg histidine hydrochloride monohydrate, 0.94 mg histidine free base, 40.03 mg arginine hydrochloride, 57.0 mg sucrose, and 0.95 mg polysorbate-80. In certain embodiments, the composition further comprises a thiol-containing antioxidant (e.g., GSH, GSSG, GSH+GSSG, cysteine, cystine, cysteine+cystine) at a concentration of 0.02 mM to 2 mM.

In certain embodiments, the composition (e.g., a pharmaceutical composition) comprises an anti-BDCA2 antibody or a BDCA2-binding fragment thereof, arginine (e.g., arginine hydrochloride) at a concentration of 50 mM to 250 mM, polysorbate-80 at a concentration of 0.02% to 0.08%, and histidine at a concentration of 10 mM to 30 mM. The composition has a pH of 5.0 to 6.5. In certain embodiments, the anti-BDCA2 antibody or BDCA2-binding fragment thereof is present in the composition at a concentration of 50 mg/ml to 225 mg/ml. In certain embodiments, the anti-BDCA2 antibody or BDCA2-binding fragment thereof of the composition comprises a VH and a VL comprising the CDRs of BIIB059 (e.g., SEQ ID NOs.: 1 or 17, 2, 3, 4, 5, and 6). In certain embodiments, the anti-BDCA2 antibody or BDCA2-binding fragment thereof of the composition comprises a VH and a VL comprising SEQ ID NOs: 7 and 8, respectively. In some embodiments, the anti-BDCA2 antibody or BDCA2-binding fragment thereof of the composition comprises a heavy chain and a light chain comprising SEQ ID NOs: 9 and 10, respectively. In certain embodiments, the composition comprises sucrose at a concentration of 1% to 10%. In certain embodiments, the composition comprises a thiol-containing antioxidant (e.g., GSH, GSSG, GSH+GSSG, cysteine, cystine, or cysteine+cystine) at a concentration of 0.02 mM to 2 mM. In one embodiment, the composition has a pH of 5.5 and comprises BIIB059 or a BIIB059-binding fragment thereof at a concentration of 150 mg/ml, sucrose at a concentration of 3%, arginine hydrochloride at a concentration of 100 mM, polysorbate-80 at a concentration of 0.05%, and histidine at a concentration of 20 mM. In another embodiment, the above-listed composition further comprises a thiol-containing antioxidant (e.g., GSH, GSSG, GSH+GSSG, cysteine, cystine, or cysteine+cystine) at a concentration of 0.02 mM to 2 mM. In a specific embodiment, the thiol-containing antioxidant is GSH at a concentration of 0.4 mM.

For subcutaneous administration, the composition (e.g., a pharmaceutical composition) may comprise higher concentration of the anti-BDCA2 antibody or BDCA2-binding fragment thereof. In one embodiment, such a composition comprises an anti-BDCA2 antibody or a BDCA2-binding fragment thereof at a concentration of 200 mg/ml; arginine (e.g., arginine hydrochloride) at a concentration of 250 mM; sucrose at a concentration of 3%; polysorbate-80 at a concentration of 0.05%; and histidine at a concentration of 20 mM. In some cases, the pH of this composition is 6.0. In some cases, the composition further comprises a thiol-containing antioxidant (e.g., GSH, GSSG, GSH+GSSG, cysteine, cystine, or cysteine+cystine) at a concentration of 0.02 mM to 2 mM. In a specific instance, the thiol-containing antioxidant is GSH at a concentration of 0.4 mM. In another specific instance, the thiol-containing antioxidant is GSSG at a concentration of 0.2 mM. In yet another specific instance, the thiol-containing antioxidant is GSH at a concentration of 0.4 mM and GSSG at a concentration of 0.2 mM. In another embodiment, such a high concentration composition comprises an anti-BDCA2 antibody or a BDCA2-binding fragment thereof at a concentration of 225 mg/ml; arginine (e.g., arginine hydrochloride) at a concentration of 250 mM; sucrose at a concentration of 1%; polysorbate-80 at a concentration of 0.05%, and histidine at a concentration of 20 mM. In some cases, the pH of this composition is 6.0. In some cases, the composition further comprises a thiol-containing antioxidant (e.g., GSH, GSSG, GSH+GSSG, cysteine, cystine, or cysteine+cystine) at a concentration of 0.02 mM to 2 mM. In a specific instance, the thiol-containing antioxidant is GSH at a concentration of 0.4 mM. In another specific instance, the thiol-containing antioxidant is GSSG at a concentration of 0.2 mM. In yet another specific instance, the thiol-containing antioxidant is GSH at a concentration of 0.4 mM and GSSG at a concentration of 0.2 mM. In another specific instance, the thiol-containing antioxidant is cysteine at a concentration of 0.4 mM. In certain embodiments, the anti-BDCA2 antibody or BDCA2-binding fragment thereof of the composition comprises a VH and a VL comprising the CDRs of BIIB059 (e.g., SEQ ID NOs.: 1 or 17, 2, 3, 4, 5, and 6). In certain embodiments, the anti-BDCA2 antibody or BDCA2-binding fragment thereof of the composition comprises a VH and a VL comprising SEQ ID NOs: 7 and 8, respectively. In some embodiments, the anti-BDCA2 antibody or BDCA2-binding fragment thereof of the composition comprises a heavy chain and a light chain comprising SEQ ID NOs: 9 and 10, respectively.

### Dosing

The anti-BDCA2 antibody (e.g., BIIB059) or BDCA2-binding fragment thereof described above can be administered to a subject, e.g., a human subject, at different doses. The anti-BDCA2 antibody (e.g., BIIB059) or BDCA2-binding fragment thereof can be administered as a fixed dose (i.e., independent of the weight of the patient), or in a mg/kg dose (i.e., a dose which varies based on the weight of the subject). Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. The treatment can continue for days, weeks, months or even years.

In one embodiment, for treating an indication described herein in an adult human subject, the dosage of the anti-BDCA2 antibody (e.g., BIIB059) or BDCA2-binding fragment thereof is a fixed dose of 25 mg. In another embodiment, the dosage of the anti-BDCA2 antibody or BDCA2-binding fragment thereof is a fixed dose of 50 mg. In another embodiment, the dosage of the anti-BDCA2 antibody or BDCA2-binding fragment thereof is a fixed dose of 150 mg. In yet another embodiment, the dosage of the anti-BDCA2 antibody or BDCA2-binding fragment thereof is a fixed dose of 450 mg.

In one embodiment, for treating an indication described herein in a pediatric human subject, the dosage of the anti-BDCA2 antibody (e.g., BIIB059) or BDCA2-binding fragment thereof is a fixed dose of 18 mg, where the child has a weight of 10 to 18 kg. In another embodiment, the dosage of the anti-BDCA2 antibody or BDCA2-binding fragment thereof is a fixed dose of 22 mg, where the child has a weight of 18.1 kg to 25 kg. In another embodiment, the dosage of the anti-BDCA2 antibody or BDCA2-binding fragment thereof is a fixed dose of 28 mg, where the child has a weight of 25.1 kg to 48 kg. In another embodiment, the dosage of the anti-BDCA2 antibody or BDCA2-binding fragment thereof is a fixed dose of 50 mg, where the child has a weight of greater than 48 kg. These doses are equivalent to an adult dose of 50 mg.

In one embodiment, for treating an indication described herein in a pediatric human subject, the dosage of the anti-BDCA2 antibody (e.g., BIIB059) or BDCA2-binding fragment thereof is a fixed dose of 40 mg, where the child has a weight of 10 to 18 kg. In another embodiment, the dosage of the anti-BDCA2 antibody or BDCA2-binding fragment thereof is a fixed dose of 56 mg, where the child has a weight of 18.1 kg to 25 kg. In another embodiment, the dosage of the anti-BDCA2 antibody or BDCA2-binding fragment thereof is a fixed dose of 80 mg, where the child has a weight of 25.1 kg to 48 kg. In another embodiment, the dosage of the anti-BDCA2 antibody or BDCA2-binding fragment thereof is a fixed dose of 150 mg, where the child has a weight of greater than 48 kg. These doses are equivalent to an adult dose of 150 mg.

The fixed doses described above may each be administered daily, every week, every 2 weeks, every 4 weeks, every 6 weeks, every 8 weeks, monthly, biweekly, weekly, or daily, as appropriate, over a period of time to encompass at least 2 doses, 3 doses, 4 doses, 5 doses, 6 doses, 7 doses, 8 doses, 9 doses, 10 doses, 12 doses, 14 doses, 16 doses, 18 doses, 20 doses, 22 doses, 24 doses or more.

In certain embodiments a fixed dose of 25 mg of the anti-BDCA2 antibody or BDCA2-binding fragment thereof is administered to a human subject every 2 weeks or every 4 weeks for a period of time determined to be beneficial for the subject by her/his healthcare provider. In some instances, a fixed dose of 25 mg of the anti-BDCA2 antibody or BDCA2-binding fragment thereof is administered to a human subject every 4 weeks. In certain embodiments, the subject is also administered a loading dose of 25 mg, 50 mg, 150 mg, or 450 mg of the anti-BDCA2 antibody or BDCA2-binding fragment thereof two weeks after the first dose of the anti-BDCA2 antibody or BDCA2-binding fragment thereof is administered to the subject. In one embodiment, the loading dose is 25 mg of the anti-BDCA2 antibody or BDCA2-binding fragment thereof. In another embodiment, the loading dose is 50 mg of the anti-BDCA2 antibody or BDCA2-binding fragment thereof. In some embodiments, the subject is administered at least 4, at least 5, at least 6, at least 7, at least 8, at least 9, or at least 10 doses of the fixed dose of 25 mg of the anti-BDCA2 antibody or BDCA2-binding fragment thereof. In some embodiments, the subject is administered 4, 5, 6, 7, 8, 9, or 10 doses of the fixed dose of 25 mg of the anti-BDCA2 antibody or BDCA2-binding fragment thereof. In some instances, the subject is administered 2 to 24, 2 to 20, 2 to 18, 2 to 16, 2 to 14, 2 to 12, 2 to 10, or 2 to 8 doses of the fixed dose of 25 mg of the anti-BDCA2 antibody or BDCA2-binding fragment thereof.

In certain embodiments a fixed dose of 50 mg of the anti-BDCA2 antibody or BDCA2-binding fragment thereof is administered to a human subject every 2 weeks or every 4 weeks for a period of time determined to be beneficial for the subject by her/his healthcare provider. In some instances, a fixed dose of 50 mg of the anti-BDCA2 antibody or BDCA2-binding fragment thereof is administered to a human subject every 4 weeks. In certain embodiments, the subject is also administered a loading dose of 25 mg, 50 mg, 150 mg, or 450 mg of the anti-BDCA2 antibody or BDCA2-binding fragment thereof two weeks after the first dose of the anti-BDCA2 antibody or BDCA2-binding fragment thereof is administered to the subject. In one embodiment, the loading dose is 50 mg of the anti-BDCA2 antibody or BDCA2-binding fragment thereof. In some embodiments, the subject is administered at least 4, at least 5, at least 6, at least 7, at least 8, at least 9, or at least 10 doses of the fixed dose of 50 mg of the anti-BDCA2 antibody or BDCA2-binding fragment thereof. In some embodiments, the subject is administered 4, 5, 6, 7, 8, 9, or 10 doses of the fixed dose of 50 mg of the anti-BDCA2 antibody or BDCA2-binding fragment thereof. In some instances, the subject is administered 2 to 24, 2 to 20, 2 to 18, 2 to 16, 2 to 14, 2 to 12, 2 to 10, or 2 to 8 doses of the fixed dose of 50 mg of the anti-BDCA2 antibody or BDCA2-binding fragment thereof.

In certain embodiments a fixed dose of 150 mg of the anti-BDCA2 antibody or BDCA2-binding fragment thereof is administered to a human subject every 2 weeks or every 4 weeks for a period of time determined to be beneficial for the subject by her/his healthcare provider. In some instances, a fixed dose of 150 mg of the anti-BDCA2 antibody or BDCA2-binding fragment thereof is administered to a human subject every 4 weeks. In certain embodiments, the subject is also administered a loading dose of 50 mg, 150 mg, or 450 mg of the anti-BDCA2 antibody or BDCA2-binding fragment thereof two weeks after the first dose of the anti-BDCA2 antibody or BDCA2-binding fragment thereof is administered to the subject. In one embodiment, the loading dose is 150 mg of the anti-BDCA2 antibody or BDCA2-binding fragment thereof. In some embodiments, the subject is administered at least 4, at least 5, at least 6, at least 7, at least 8, at least 9, or at least 10 doses of the fixed dose of 150 mg of the anti-BDCA2 antibody or BDCA2-binding fragment thereof. In some embodiments, the subject is administered 4, 5, 6, 7, 8, 9, or 10 doses of the fixed dose of 150 mg of the anti-BDCA2 antibody or BDCA2-binding fragment thereof. In some instances, the subject is administered 2 to 24, 2 to 20, 2 to 18, 2 to 16, 2 to 14, 2 to 12, 2 to 10, or 2 to 8 doses of the fixed dose of 150 mg of the anti-BDCA2 antibody or BDCA2-binding fragment thereof.

In certain embodiments a fixed dose of 450 mg of the anti-BDCA2 antibody or BDCA2-binding fragment thereof is administered to a human subject every 2 weeks or every 4 weeks for a period of time determined to be beneficial for the subject by her/his healthcare provider. In some instances, a fixed dose of 450 mg of the anti-BDCA2 antibody or BDCA2-binding fragment thereof is administered to a human subject every 4 weeks. In certain embodiments, the subject is also administered a loading dose of 50 mg, 150 mg, or 450 mg of the anti-BDCA2 antibody or BDCA2-binding fragment thereof two weeks after the first dose of the anti-BDCA2 antibody or BDCA2-binding fragment thereof is administered to the subject. In one embodiment, the loading dose is 450 mg of the anti-BDCA2 antibody or BDCA2-binding fragment thereof. In some embodiments, the subject is administered at least 4, at least 5, at least 6, at least 7, at least 8, at least 9, or at least 10 doses of the fixed dose of 450 mg of the anti-BDCA2 antibody or BDCA2-binding fragment thereof. In some embodiments, the subject is administered 4, 5, 6, 7, 8, 9, or 10 doses of the fixed dose of 450 mg of the anti-BDCA2 antibody or BDCA2-binding fragment thereof. In some instances, the subject is administered 2 to 24, 2 to 20, 2 to 18, 2 to 16, 2 to 14, 2 to 12, 2 to 10, or 2 to 8 doses of the fixed dose of 450 mg of the anti-BDCA2 antibody or BDCA2-binding fragment thereof.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual. A therapeutically effective amount is also one in which any toxic, or detrimental effects, of the composition is outweighed by the therapeutically beneficial effects. In one embodiment, the therapeutically effective amount of the anti-BDCA2 antibody or BDCA2-binding fragment thereof is 25 mg. In another embodiment, the therapeutically effective amount of the anti-BDCA2 antibody or BDCA2-binding fragment thereof is 50 mg. In another embodiment, the therapeutically effective amount of the anti-BDCA2 antibody or BDCA2-binding fragment thereof is 150 mg. In yet another embodiment, the therapeutically effective amount of the anti-BDCA2 antibody or BDCA2-binding fragment thereof is 450 mg. In one embodiment, the therapeutically effective amount of the anti-BDCA2 antibody or BDCA2-binding fragment thereof for a pediatric human subject (e.g., a subject 21 years of age or less, a subject 18 years of age or less, or a subject 16 years of age or less) is 18 mg, 22 mg, 28 mg, 40 mg, 50 mg, 56 mg, 80 mg, or 150 mg.

In some instances, the anti-BDCA2 antibody or BDCA2-binding compositions described above are administered to the subject at a dose of 25 mg. In other instances, the anti-BDCA2 antibody or BDCA2-binding compositions described above are administered to the subject at a dose of 50 mg. In yet other instances, the anti-BDCA2 antibody or BDCA2-binding compositions described above are administered to the subject at a dose of 150 mg. In certain instances, the anti-BDCA2 antibody or BDCA2-binding compositions described above are administered to the subject at a dose of 450 mg.

For pediatric human subjects (e.g., a subject 21 years of age or less, a subject 18 years of age or less, or a subject 16 years of age or less), to achieve the equivalent of a 50 mg adult dose of the anti-BDCA2 antibody or BDCA2-binding fragment, the dose is determined based on the weight of the child as follows:

| Weight Category | Dose to be Administered |
|---|---|
| 10 to 18 kg | 18 mg every four weeks |
| 18.1 to 25 kg | 22 mg every four weeks |
| 25.1 to 48 kg | 28 mg every four weeks |
| greater than 48 kg | 50 mg every four weeks. |

For pediatric human subjects, to achieve the equivalent of a 150 mg adult dose of the anti-BDCA2 antibody or BDCA2-binding compositions described above, the dose is determined based on the weight of the child as follows:

| Weight Category | Dose to be Administered |
|---|---|
| 10 to 18 kg | 40 mg every four weeks |
| 18.1 to 25 kg | 56 mg every four weeks |
| 25.1 to 48 kg | 80 mg every four weeks |
| greater than 48 kg | 150 mg every four weeks. |

The route and/or mode of administration of the anti-BDCA2 antibody or BDCA2-binding fragment thereof can be tailored for the individual subject. For many applications, the route of administration is one of: subcutaneous injection (SC), intravenous injection or infusion (IV), intraperitoneal administration (IP), or intramuscular injection. In one embodiment, the route of administration is subcutaneous. In another embodiment, the route of administration is intravenous.

Pharmaceutical compositions that comprise the anti-BDCA2 antibody or BDCA2-binding fragment thereof alone or in combination with non-BDCA2 antibody agent(s) can be administered with a medical device. The device can be designed with features such as portability, room temperature storage, and ease of use so that it can be used in emergency situations, e.g., by an untrained subject or by emergency personnel in the field, removed to medical facilities and other medical equipment. The device can include, e.g., one or more housings for storing pharmaceutical preparations that include the anti-BDCA2 antibody or BDCA2-binding fragment thereof, and can be configured to deliver one or more unit doses of the blocking agent.

For example, the pharmaceutical composition can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

In one embodiment, the anti-BDCA2 antibody or BDCA2-binding fragment thereof is administered to a human subject with a syringe. In another embodiment, the anti-BDCA2 antibody or BDCA2-binding fragment thereof is administered to a human subject with a pump for subcutaneous delivery. In some embodiments, the anti-BDCA2 antibody or BDCA2-binding fragment thereof is administered to a human subject with an autoinjector. In other embodiments, the anti-BDCA2 antibody or BDCA2-binding fragment thereof is administered to a human subject with a subcutaneous large volume injector.

This disclosure provides a pump or syringe comprising a sterile preparation of an anti-BDCA2 antibody (e.g., BIIB059) or BDCA2-binding fragment thereof. The syringe or pump can be adapted for subcutaneous administration of the anti-BDCA2 antibody or BDCA2-binding fragment thereof. In some cases, the syringe or pump delivers a fixed doses(s) (e.g., 18 mg, 22 mg, 28 mg, 40 mg, 50 mg, 56 mg, 80 mg, 150 mg, 450 mg) of the anti-BDCA2 antibody or BDCA2-binding fragment thereof.

The disclosure also provides a pump, syringe, or injector (e.g., autoinjector, subcutaneous large volume injector) comprising a sterile preparation of the pharmaceutical compositions described above. The syringe or pump can be adapted for subcutaneous administration of the pharmaceutical compositions comprising the anti-BDCA2 antibody or BDCA2-binding fragment thereof. In some instances, the syringe or pump delivers a fixed doses(s) (e.g., 18 mg, 22 mg, 28 mg, 40 mg, 50 mg, 56 mg, 80 mg, 150 mg, 450 mg) of the anti-BDCA2 antibody or BDCA2-binding fragment thereof.

### Methods of Treatment

An anti-BDCA2 antibody or BDCA2-binding fragment thereof described herein can be used to treat or prevent a variety of immunological disorders, such as inflammatory and autoimmune disorders. Anti-BDCA2 antibodies or BDCA2-binding fragments thereof can disable or deplete pDCs, and/or inhibit inflammatory cytokines and chemokines produced by pDCs, and/or downregulate CD32a, and/or inhibiting immune complex stimulation of pDCs, and/or downregulate or cause shedding of CD62L. The anti-BDCA2 antibodies or BDCA2-binding fragment thereof of this disclosure can be combined with an antimalarial agent (e.g., HCQ) for improved therapeutic effects in the treatment of inflammatory and autoimmune disorders. Anti-BDCA2 antibodies can be used to reduce levels of cytokines and chemokines such as: type I interferons, type III interferons, IL-6, TNF-α, MIP1-α and MIP1-β, CCL5, and IP-10. Type I IFNs constitute a multiple-member family of cytokines, including 13 IFN-α subtypes, IFN-β, -ε, -κ, -ω, -δ and -τ. (Theofilopoulos, Annu. Rev. Immunol., 23:307-36 (2005)). Type III interferons consist of three IFN-λ molecules called IFN-λ1, IFN-λ2 and IFN-λ3 (also referred to as IL29, IL28A and IL28B, respectively). By depleting and/or dampening pDC function, the anti-BDCA2 antibodies described herein provide a more robust treatment approach than treatments attempting to reduce specific IFN subtypes with neutralizing antibodies. In addition, the pDC-focused treatment approach of the anti-BDCA2 antibodies is more selective and potentially safer than global blockade of the IFN response. For example, anti-BDCA2 antibodies described herein effectively eliminate pDC-derived type I IFNs while maintaining other sources of IFN that could be necessary in the event of viral infections.

This disclosure provides methods of treating BDCA2-associated disorders using the antibodies and compositions described herein. Non-limiting examples of BDCA2-associated disorders include SLE, CLE, DLE, lupus nephritis, systemic sclerosis (scleroderma), morphea, psoriasis, rheumatoid arthritis, inflammatory bowel disease (IBD), dermatomyositis, polymyositis, type I diabetes, and cytokine release syndrome. In some embodiments, the anti-BDCA2 antibodies and compositions described herein can be used to treat a lupus disorder (e.g., SLE, CLE, and DLE).

In one embodiment, the disclosure features a method of treating SLE (e.g., moderate or severe lupus) in a human subject in need thereof. The method involves administering to a human subject in need thereof a therapeutically effective amount of an anti-BDCA2 antibody or BDCA2-binding fragment. In certain instances, the subject is administered the pharmaceutical compositions described herein to provide a dose of 50 mg, 150 mg, or 450 mg of the anti-BDCA2 antibody or BDCA2-binding fragment. In certain instances, when the subject is a pediatric subject (e.g., a subject 21 years of age or less, a subject 18 years of age or less, or a subject 16 years of age or less), the subject is administered the pharmaceutical compositions described herein to provide a dose of 18 mg, 22 mg, 28 mg, 40 mg, 50 mg, 56 mg, 80 mg, or 150 mg of the anti-BDCA2 antibody or BDCA2-binding fragment. The dose is chosen based on the weight of the child as detailed above. In some instances, the subject is administered at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, of 12 doses. In certain instances, the subject is also administered a loading dose of 50 mg, 150 mg, or 450 mg of the anti-BDCA2 antibody or BDCA2-binding fragment about 2 weeks after the administration of the first dose of the anti-BDCA2 antibody or BDCA2-binding fragment. In one embodiment, the subject with SLE is administered a fixed dose of 50 mg of the anti-BDCA2 antibody or BDCA2-binding fragment and a loading dose of 50 mg of the anti-BDCA2 antibody or BDCA2-binding fragment at 2 weeks after the administration of the first dose of the anti-BDCA2 antibody or BDCA2-binding fragment. In another embodiment, the subject with SLE is administered a fixed dose of 150 mg of the anti-BDCA2 antibody or BDCA2-binding fragment and a loading dose of 150 mg of the anti-BDCA2 antibody or BDCA2-binding fragment at 2 weeks after the administration of the first dose of the anti-BDCA2 antibody or BDCA2-binding fragment. In yet another embodiment, the subject with SLE is administered a fixed dose of 450 mg of the anti-BDCA2 antibody or BDCA2-binding fragment and a loading dose of 450 mg of the anti-BDCA2 antibody or BDCA2-binding fragment at 2 weeks after the administration of the first dose of the anti-BDCA2 antibody or BDCA2-binding fragment.

The disclosure also features a method of treating cutaneous lupus erythematosus (with or without SLE) in a human subject in need thereof. The method involves administering to a human subject in need thereof a therapeutically effective amount of an anti-BDCA2 antibody or BDCA2-binding fragment. In certain instances, the subject is administered the pharmaceutical compositions described herein to provide a dose of 50 mg, 150 mg, or 450 mg of the anti-BDCA2 antibody or BDCA2-binding fragment. In certain instances, when the subject is a pediatric subject (e.g., a subject 21 years of age or less, a subject 18 years of age or less, or a subject 16 years of age or less), the subject is administered the pharmaceutical compositions described herein to provide a dose of 18 mg, 22 mg, 28 mg, 40 mg, 50 mg, 56 mg, 80 mg, or 150 mg of the anti-BDCA2 antibody or BDCA2-binding fragment. The dose is chosen based on the weight of the child as detailed above. In some instances, the subject is administered at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, of 12 doses. In certain instances, the subject is also administered a loading dose of 50 mg, 150 mg, or 450 mg of the anti-BDCA2 antibody or BDCA2-binding fragment about 2 weeks after the administration of the first dose of the anti-BDCA2 antibody or BDCA2-binding fragment. In one embodiment, the subject with CLE (with or without SLE) is administered a fixed dose of 50 mg of the anti-BDCA2 antibody or BDCA2-binding fragment and a loading dose of 50 mg of the anti-BDCA2 antibody or BDCA2-binding fragment at 2 weeks after the administration of the first dose of the anti-BDCA2 antibody or BDCA2-binding fragment. In another embodiment, the subject with CLE (with or without SLE) is administered a fixed dose of 150 mg of the anti-BDCA2 antibody or BDCA2-binding fragment and a loading dose of 150 mg of the anti-BDCA2 antibody or BDCA2-binding fragment at 2 weeks after the administration of the first dose of the anti-BDCA2 antibody or BDCA2-binding fragment. In yet another embodiment, the subject with CLE (with or without SLE) is administered a fixed dose of 450 mg of the anti-BDCA2 antibody or BDCA2-binding fragment and a loading dose of 450 mg of the anti-BDCA2 antibody or BDCA2-binding fragment at 2 weeks after the administration of the first dose of the anti-BDCA2 antibody or BDCA2-binding fragment.

The disclosure also provides a method of treating discoid lupus erythematosus (with or without SLE) in a human subject in need thereof. The method involves administering to a human subject in need thereof a therapeutically effective amount of an anti-BDCA2 antibody or BDCA2-binding fragment. In certain instances, the subject is administered the pharmaceutical compositions described herein to provide a dose of 50 mg, 150 mg, or 450 mg of the anti-BDCA2 antibody or BDCA2-binding fragment. In certain instances, when the subject is a pediatric subject (e.g., a subject 21 years of age or less, a subject 18 years of age or less, or a subject 16 years of age or less), the subject is administered the pharmaceutical compositions described herein to provide a dose of 18 mg, 22 mg, 28 mg, 40 mg, 50 mg, 56 mg, 80 mg, or 150 mg of the anti-BDCA2 antibody or BDCA2-binding fragment. The dose is chosen based on the weight of the child as detailed above. In some instances, the subject is administered at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, of 12 doses. In certain instances, the subject is also administered a loading dose of 50 mg, 150 mg, or 450 mg of the anti-BDCA2 antibody or BDCA2-binding fragment about 2 weeks after the administration of the first dose of the anti-BDCA2 antibody or BDCA2-binding fragment. In one embodiment, the subject with discoid lupus (with or without SLE) is administered a fixed dose of 50 mg of the anti-BDCA2 antibody or BDCA2-binding fragment and a loading dose of 50 mg of the anti-BDCA2 antibody or BDCA2-binding fragment at 2 weeks after the administration of the first dose of the anti-BDCA2 antibody or BDCA2-binding fragment. In another embodiment, the subject with discoid lupus (with or without SLE) is administered a fixed dose of 150 mg of the anti-BDCA2 antibody or BDCA2-binding fragment and a loading dose of 150 mg of the anti-BDCA2 antibody or BDCA2-binding fragment at 2 weeks after the administration of the first dose of the anti-BDCA2 antibody or BDCA2-binding fragment. In yet another embodiment, the subject with discoid lupus (with or without SLE) is administered a fixed dose of 450 mg of the anti-BDCA2 antibody or BDCA2-binding fragment and a loading dose of 450 mg of the anti-BDCA2 antibody or BDCA2-binding fragment at 2 weeks after the administration of the first dose of the anti-BDCA2 antibody or BDCA2-binding fragment.

In one embodiment, the disclosure features a method of treating cytokine release syndrome and/or cytokine storms in a human subject in need thereof. Cytokine release syndrome (CRS) is a common immediate complication occurring with the use of T cell-engaging therapies (e.g., chimeric antigen receptor-modified T cell (CART) therapy). Severe cases of this disorder are known as cytokine storms. CRS is a symptom complex associated with the use of many monoclonal antibodies. Commonly referred to as an infusion reaction, CRS results from the release of cytokines from cells targeted by the antibody as well as immune effector cells recruited to the area. The antibodies bind to the T cell receptor, activating the T cells before they are destroyed. The cytokines released by the activated T cells produce a type of systemic inflammatory response similar to that found in severe infection. When cytokines are released into the circulation, the subject can develop systemic symptoms such as fever, nausea, chills, hypotension, tachycardia, asthenia, headache, rash, scratchy throat, and dyspnea. In most cases, the symptoms are mild to moderate in severity and can be managed relatively easily. However, some patients can experience severe, life-threatening reactions that result from massive release of cytokines. Severe reactions occur more commonly during the first infusion in patients with hematologic malignancies who have not received prior chemotherapy. Severe reactions are marked by their rapid onset and the acuity of associated symptoms. Massive cytokine release is an oncologic emergency and can lead to life-threatening complications. The method of treating CRS involves administering to a human subject in need thereof an anti-BDCA2 antibody or BDCA2-binding fragment. In certain instances, the subject is administered the pharmaceutical compositions described herein to provide a dose of 50 mg, 150 mg, or 450 mg of the anti-BDCA2 antibody or BDCA2-binding fragment. In certain instances, when the subject is a pediatric subject (e.g., a subject 21 years of age or less, a subject 18 years of age or less, or a subject 16 years of age or less), the subject is administered the pharmaceutical compositions described herein to provide a dose of 18 mg, 22 mg, 28 mg, 40 mg, 50 mg, 56 mg, 80 mg, or 150 mg of the anti-BDCA2 antibody or BDCA2-binding fragment. The dose is chosen based on the weight of the child as detailed above. In some instances, the subject is administered at least 2, at least 3, at least 4, at least 5, at least 6, at least 7 at least 8, at least 9, at least 10 doses, at least 11 doses, at least 12 doses, or 2, 3, 4, 5, 6, 7, 8, 9, 10, 11, of 12 doses. In certain instances, the subject is also administered a loading dose of 50 mg, 150 mg, or 450 mg of the anti-BDCA2 antibody or BDCA2-binding fragment about 2 weeks after the administration of the first dose of the anti-BDCA2 antibody or BDCA2-binding fragment. In one embodiment, the subject with CRS is administered a fixed dose of 50 mg of the anti-BDCA2 antibody or BDCA2-binding fragment and a loading dose of 50 mg of the anti-BDCA2 antibody or BDCA2-binding fragment at 2 weeks after the administration of the first dose of the anti-BDCA2 antibody or BDCA2-binding fragment. In another embodiment, the subject with CRS is administered a fixed dose of 150 mg of the anti-BDCA2 antibody or BDCA2-binding fragment and a loading dose of 150 mg of the anti-BDCA2 antibody or BDCA2-binding fragment at 2 weeks after the administration of the first dose of the anti-BDCA2 antibody or BDCA2-binding fragment. In yet another embodiment, the subject with CRS is administered a fixed dose of 450 mg of the anti-BDCA2 antibody or BDCA2-binding fragment and a loading dose of 450 mg of the anti-BDCA2 antibody or BDCA2-binding fragment at 2 weeks after the administration of the first dose of the anti-BDCA2 antibody or BDCA2-binding fragment. In certain instances, the human subject has, is scheduled to, or is undergoing CART therapy (e.g., CART-19 therapy). In certain instances, the human subject has, is scheduled to, or is undergoing therapy with an anti-T cell antibody (e.g., ATG, OKT3, TGN1412) or bispecific antibody (e.g., blinatumomab). In certain instances, the subject has, is scheduled to, or is undergoing therapy with an anti-CD20 antibody (e.g., rituximab). In certain instances, the human subject being treated for CRS is also administered a corticosteroid (e.g., hydrocortisone) and/or an anti-histamine (e.g., chlorphenamine) simultaneously, separately, or sequentially during the treatment with the anti-BDCA2 antibody or BDCA2-binding fragment thereof. In some instances, the subject is also administered an agent that inhibits IL-6 simultaneously, separately, or sequentially during the treatment with the anti-BDCA2 antibody or BDCA2-binding fragment thereof. The agent that inhibits IL-6 may be an anti-IL-6 antibody or IL6-binding fragment thereof, an IL6 receptor (IL6R) antagonist (e.g., tocilizumab or a soluble IL6R).

In one embodiment in all of the above-described methods of treatment, the anti-BDCA2 antibody or BDCA2-binding fragment thereof comprises the three heavy chain variable domain CDRs and the three light chain variable domain CDRs of BIIB059. In another embodiment, the anti-BDCA2 antibody or BDCA2-binding fragment comprises the amino acid sequences set forth in SEQ ID NOs.: 1-6. In another embodiment, the anti-BDCA2 antibody or BDCA2-binding fragment comprises the amino acid sequences set forth in SEQ ID NOs.: 12-16. In yet another embodiment, the anti-BDCA2 antibody or BDCA2-binding fragment comprises the amino acid sequences set forth in SEQ ID NOs.: 18-22. In a further embodiment, the anti-BDCA2 antibody or BDCA2-binding fragment comprises the amino acid sequences set forth in SEQ ID NOs.: 24-28. In one embodiment, the anti-BDCA2 antibody or BDCA2-binding fragment thereof comprises a VH CDR1 comprising or consisting of the amino acid sequence set forth in SEQ ID NO.:1 or 17, a VH CDR2 comprising or consisting of the amino acid sequence set forth in SEQ ID NO.: 2; and a VH CDR3 comprising or consisting of the amino acid sequence set forth in SEQ ID NO. 3; and a VL CDR1 comprising or consisting of the amino acid sequence set forth in SEQ ID NO.:4, a VL CDR2 comprising or consisting of the amino acid sequence set forth in SEQ ID NO.: 5; and a VL CDR3 comprising or consisting of the amino acid sequence set forth in SEQ ID NO. 6.

In some embodiments in all of the above-described methods of treatment, the anti-BDCA2 antibody or antigen-binding fragment thereof selectively binds to the ectodomain of human BDCA2 and comprises (i) a VH domain that is at least 70%, 75%, 80%, 85%, 90%, 91%, 92%, 93%, 94%, 95%, 96%, 97%, 98%, 99% or more identical to the amino acid sequence of the VH domain of BIIB059 (SEQ ID NO:7), and/or (ii) a VL domain that is at least 70%, 75%, 80%, 85%, 90%, 91%, 92%, 93%, 94%, 95%, 96%, 97%, 98%, 99% or more identical to the amino acid sequence of the VL domain of BIIB059 (SEQ ID NO:8); or differs at least at 1 to 5 amino acid residues, but at fewer than 40, 30, 20, 15, or 10, residues, from SEQ ID NO:7 and/or SEQ ID NO:8. In certain instances, these anti-BDCA2 antibodies or BDCA2-binding fragments (i) bind human or cynomolgus monkey BDCA2 but do not significantly bind BDCA2 from phylogenetic species below primates; and/or (ii) inhibit TLR7/TLR9-induced type I interferon and other cytokine or chemokine production by human pDCs; and/or (iii) mediate internalization of BDCA2 from the surface of pDCs; and/or (iv) downregulate CD32a and/or CD62L from the surface of pDCs; and/or (v) deplete pDCs in vitro by ADCC or CDC.

In certain embodiments in all of the above-described methods of treatment, the anti-BDCA2 antibody or antigen-binding fragment thereof selectively binds to the ectodomain of human BDCA2 and comprises (i) a HC that is at least 70%, 75%, 80%, 85%, 90%, 91%, 92%, 93%, 94%, 95%, 96%, 97%, 98%, 99% or more identical to the amino acid sequence of SEQ ID NO:9, and/or (ii) a LC that is at least 70%, 75%, 80%, 85%, 90%, 91%, 92%, 93%, 94%, 95%, 96%, 97%, 98%, 99% or more identical to the amino acid sequence of SEQ ID NO:10; or differs at least at 1 to 5 amino acid residues, but at fewer than 40, 30, 20, 15, or 10, residues, from SEQ ID NO:9 and/or SEQ ID NO:10.

The following are examples of the practice of the invention. They are not to be construed as limiting the scope of the invention in any way.

### EXAMPLES

### Example 1: Assessing Viscosity of Anti-BDCA2 Antibody Formulations

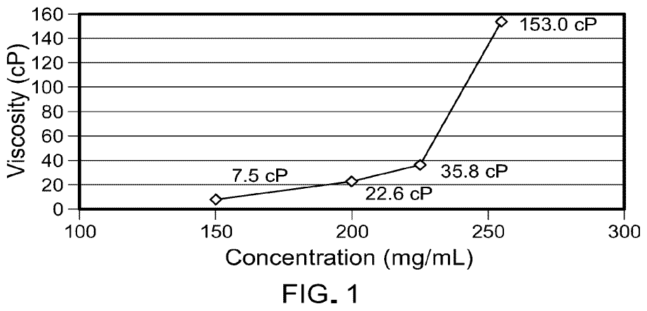

To develop a high concentration anti-BDCA2 antibody formulation, the highest concentration of antibody that could be used was determined. The antibody formulation used in these studies comprised BIIB059, 10 mM citrate buffer, 140 mM Arg·HCl and 0.05% PS80. The formulation had a pH of 6.0. The highest concentration in these studies would be limited by viscosity and the limit imposed by the large volume subcutaneous pump: 50 cP. The viscosity was measured in the low concentration formulation (FIG. 1). It was found that the threshold viscosity was crossed between 225 and 250 mg/mL and 225 mg/mL was chosen as the highest concentration for anti-BDCA2 antibody formulations.

### Example 2: Testing Different Excipients and Conditions for the Antibody Formulation

Initially, very high aggregation rates were observed at 40° C., as well as visible particles and significant sub-visible particulate loads, in the antibody formulation of Example 1. Several causative factors were identified:
1. Behavior at 40° C. is not apparently predictive of that at 5° C.
2. Process-related stresses, e.g., during UF/DF, can cause aggregates to form. Processing in the presence of excipients prevents this from occurring
3. Different drug substance batches used had different starting levels of HMW which may influence subsequent aggregation
4. The protein appeared at least moderately light sensitive.
5. There may be a link with oxidation occurring.

Material was therefore prepared in the presence of at least minimal excipients, before spiking with any further excipients. The formulations tested on stability are shown in Table 2.

TABLE 2
Initial formulation studies

| | Protein | | | |
|---|---|---|---|---|
| | concentration | | | |
| Study | (mg/mL) | Buffer | pH | Excipient |
| 1 | 150, 200 and 225 | His, | 5.5, 6.0, | 140 mM Arg · HCl |
| | | Citrate | 6.5 | |
| 2 | 225 | Citrate | 6.0 | 70 mM Arg |
| | | | | 300 mM Arg |
| | | | | 7% sucrose |
| | | | | 300 mM Pro |
| | | | | 150 mM Arg, 10 mM Met |
| | | | | 70 mM Arg, 70 mM Glu |
| | | | | 140 mM NaCl |
| | | | | 1% hydroxypropyl β- |
| | | | | cyclodextrin |
| | | | | 1% succinyl β-cyclodextrin |

### Study 1

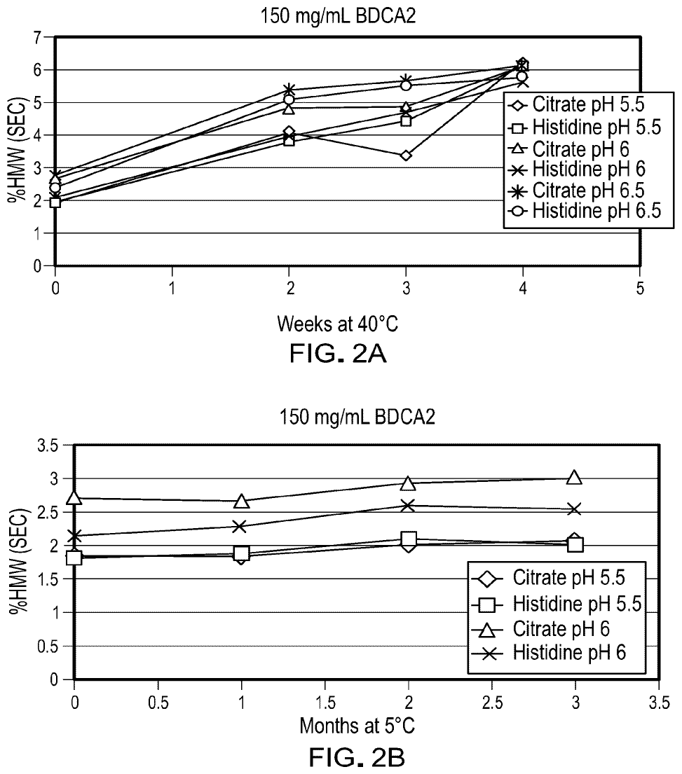

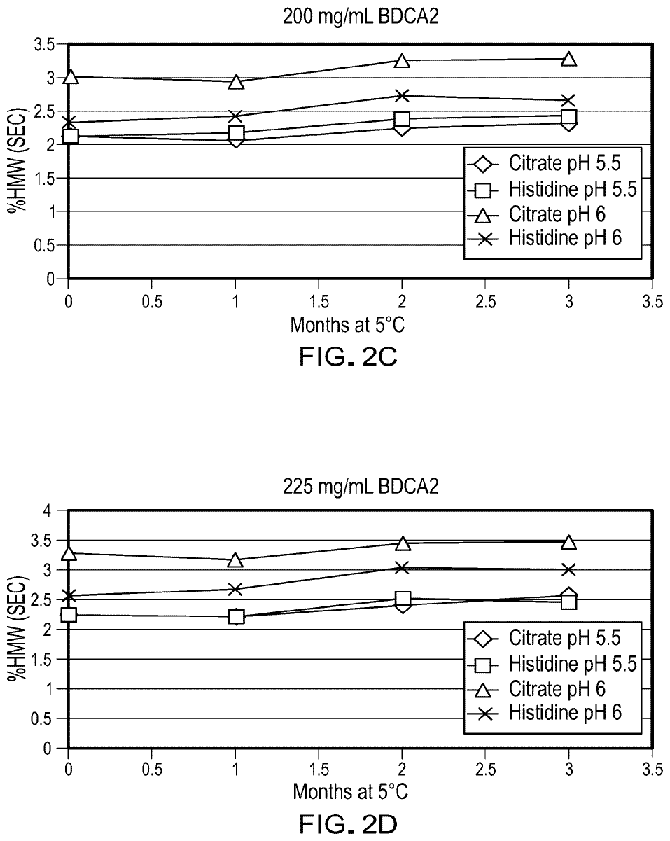

In Study 1, although high aggregation was observed at 40° C., excellent stability was observed at 5° C., with no significant increases in high molecular weight species (HMW) over 3 months at concentrations up to 225 mg/mL. Further analysis of the data showed that lower pH resulted in lower aggregation, and histidine was better than citrate (FIG. 2).

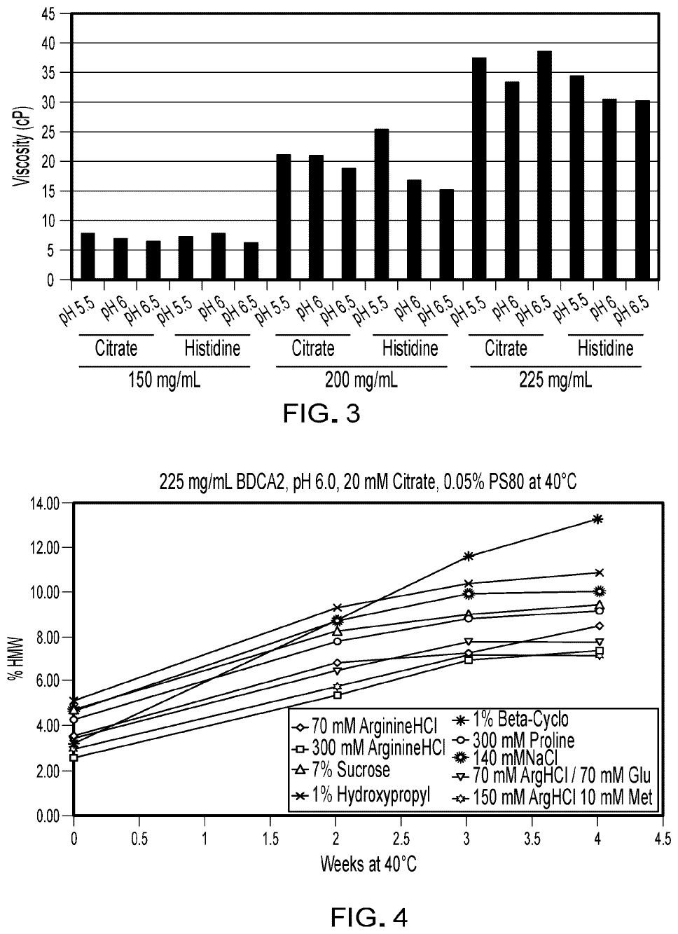

Visible particles could be observed after incubation at 40° C. at the higher pH, while sub-visible particulate counts by micro-flow imaging (MFI) remained acceptable. A similar trend was observed after 3 months at 5° C., although fewer particles could be seen. The viscosity in these formulations increased with concentration. A weak dependency on buffer and pH could also be observed, although this effect was small (FIG. 3).

Experiments were also run to determine whether the Tween levels used, 0.05% PS80, were still adequate at high concentration of the anti-BDCA2 antibody. Appearance, MFI, and size exclusion chromatography (SEC) all showed that at levels of 0.02% PS80 and above, no additional protection against agitation was obtained. Maintaining the target concentration at 0.05% PS80 was therefore determined to be adequate.

### Study 2

This second study looked at different excipients and some excipient combinations (see, Table 2).

At 40° C., high aggregation was again observed, although some excipients, notably Arg·HCl, provided a clear advantage in a concentration-dependent manner (FIG. 4).

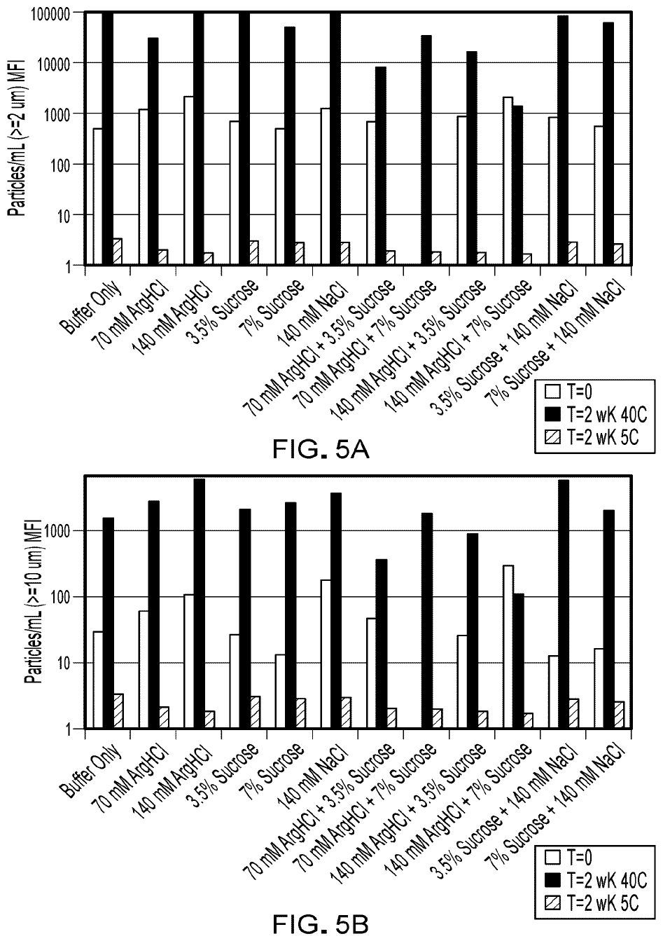

In considering the viscosity of these formulations, Arg·HCl was again seen to be an advantage as it lowers the viscosity in a concentration-dependent manner. The Arg containing solutions did have a propensity for forming visible particles after incubation at 40° C. Surprisingly, sucrose prevented the formation of visible particles (Table 3). Sucrose also lowered the counts of sub-visible-particulates (FIG. 5).

TABLE 3
Viscosity (at time zero) and visible particles after incubation at
40° C. for 1 month. Formulations were 20 mM Citrate pH
6.0, 0.05% PS80 with the additional excipients as shown.

| | Viscosity at | Visible particles after 1 month |
|---|---|---|
| Excipient | 225 mg/mL | at 40° C. |
| 70 mM Arg · HCl | 47.4 | gross white/opaque visible |
| | | particles created by swirling |
| 300 mM Arg · HCl | 26.1 | gross visible particles created |
| | | by swirling |
| 7% sucrose | 168 | no visible particles |
| 1% hydroxypropyl β- | 167 | no visible particles |
| cyclodextrin | | |
| 1% succinyl β-cyclodextrin | 202 | no visible particles |
| 300 mM Proline | 104 | no visible particles |
| 140 mM NaCl | 59.1 | no visible particles |
| 70 mM Arg · HCl/70 mM | 45.9 | no visible particles |
| Glu | | |
| 150 mM Arg · HCl/10 mM | 33.5 | gross visible particles created |
| Met | | by swirling |

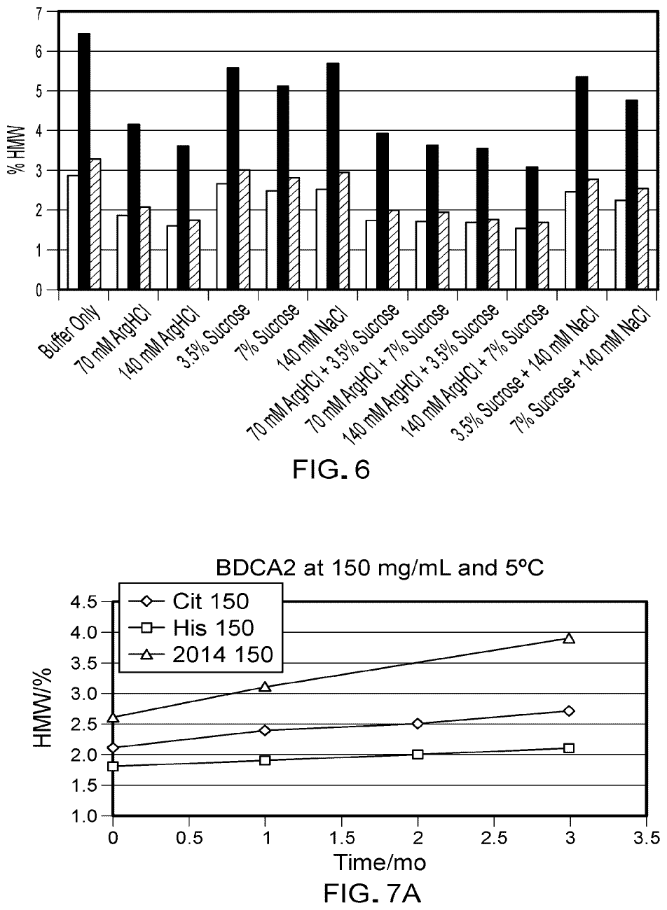

Based on these results, a developmental stability study using the combination of sucrose and Arg·HCl was started to see if the combination would result in lower aggregation, good viscosity, and no particle formation. No visible particulates could be observed after incubation at 40° C. Interestingly, the combination of sucrose and Arg·HCl also significantly lowered the sub-visible particulate count (FIG. 5). Although the number of particulates in 70 mM Arg·HCl was quite low, the addition of sucrose, surprisingly, further lowered the particle count (FIG. 5). The presence of sucrose did not significantly affect the formation of aggregates (FIG. 6). Additional data out to 6 weeks at 5° C. continued this trend and showed acceptable stability in the Arg·HCl/sucrose combination formulations. At 200 mg/mL, the viscosity of 70 mM Arg·HCl with 3.5% sucrose was 22.5 cP; with 7% sucrose the viscosity was 23.5 cP.

Combining the results from Study 1 and Study 2, a number of observations led to the proposal of a new “best” high concentration formulation (Table 4). This “best” formulation is referred to as Formulation 2 in Examples 3 and 4.

TABLE 4
Details of a proposed “best” formulation, combining data from Study 1 and Study 2

| | Original 50 mg/mL | Proposed new | |
|---|---|---|---|
| | formulation | formulation | Rationale |
| Buffer | 10 mM Citrate | 20 mM His | Increase buffering capacity |
| | | | Better stability in His |
| pH | pH 6.0 | pH 5.5 | Lower aggregation at lower pH |
| [Arg · HCl] | 140 mM | 100 mM | Balance osmolality vs. lower viscosity and |
| | | | aggregation |
| [Sucrose] | — | 3% | Reduce visible particle and sub-visible |
| | | | particulate formation |
| PS80 | 0.05% | 0.05% | No change needed |
| concentration | | | |

Because there was a history of aggregation, sub-visible particulates, and visible particles in anti-BDCA2 formulations, it was decided that the anti-BDCA2 antibody be formulated at 150 mg/mL.

### Example 3: Comparing Aggregation in Anti-BDCA2 Antibody Formulations

A 50 mg/ml, anti-BDCA2 antibody (BIIB059) formulation formulated in 10 mM Citrate, 150 mM Arg·HCl, 0.05% PS80, pH 6.0 was subjected to concentration by ultrafiltration/diafiltration. Two different concentrated formulations were created: Formulation 1: 150 mg/ml BIIB059, 20 mM citrate, 140 mM Arg·HCl, 0.05% PS80, pH 6.0; and Formulation 2: 150 mg/ml BIIB059, 20 mM histidine, 100 mM Arg·HCl, 3% sucrose, 0.05% PS80, pH 5.5. With this format it was possible to explore high concentration in two different formulations.

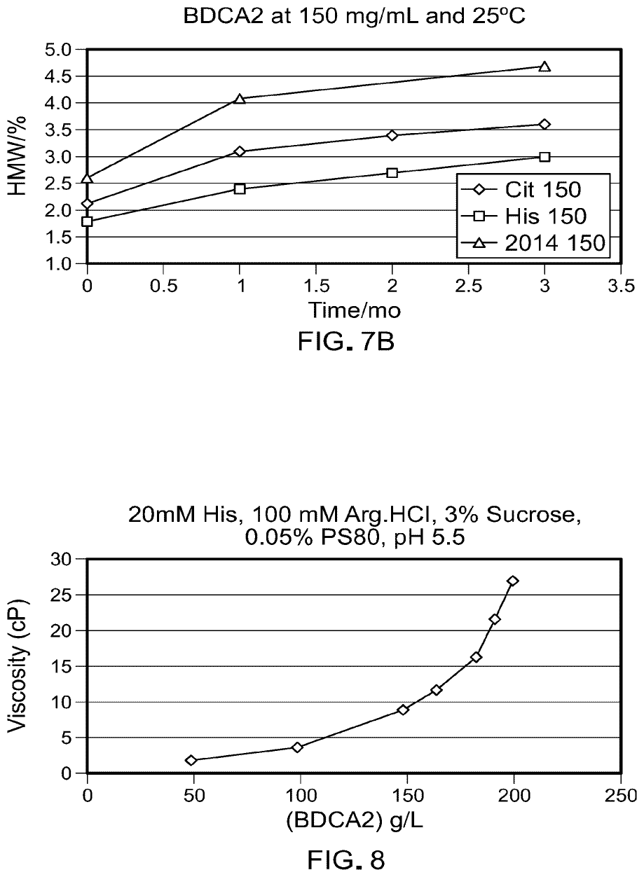

Interestingly, although the Formulation 2 material was concentrated and reprocessed from the Citrate/Arg buffer, Formulation 2 (with the His/Sucrose/Arg excipients) showed lower levels of starting aggregate (FIG. 7). The aggregation rate of this material was also lower (Table 5).

TABLE 5
Aggregation rates (% HMW increase per month) comparing
Formulation 1 and 2

| | Formulation 2 | Formulation 1 |
|---|---|---|
| Formulation | 150 mg/mL, | 150 mg/mL, |
| | His/Arg/Sucrose | Cit/Arg |
| 5° C. aggregation rate | 0.10 | 0.20 |
| 25° C. aggregation rate | 0.40 | 0.50 |
| 40° C. aggregation rate | 2.53 | 2.00 |

Based on the observed, starting % HMW (FIG. 7), the rate at of aggregation at 5° C. (Table 5) and the increase in HMW after 1 month at 25° C. (FIG. 7), it was possible to predict the shelf life of each product: i.e., the time it takes to reach 5% HMW, the typical specification threshold for early stage products. The predicted shelf life for the Formulation 1 was 9.5 months, while that for Formulation 2 was 26 months (this is likely to even be an underestimate, as the 5° C. aggregation rate was based on the first three months of data where aggregation was fastest, and 1 month room temperature was likely much beyond what the product might actually be subjected to). In sum, the data show that Formulation 2 affords significantly increased stability against aggregation as compared to Formulation 1.

### Example 4: Viscosity of Anti-BDCA2 Antibody Formulation 2

The viscosity of Formulation 2 was then measured. As can be seen in FIG. 8, the viscosity profile was amenable to incorporating this formulation into a device. The 10 cP threshold for an autoinjector was not crossed until ˜155 mg/mL, suggesting material of up to ˜140 mg/mL could go into this device. The 50 cP threshold had not been crossed at concentrations as high as 200 mg/mL, suggesting the possibility of going up to this concentration should a subcutaneous large volume injector be required.

### Example 5: Rationale for Dosing Regimen

Dosing regimens were selected based on safety, pharmacokinetics (PK), PK-BDCA2 internalization relationship, and extrapolated inhibitory potency (concentration resulting in 90% inhibition of response [IC90]) of pDC IFNα production.

Single IV doses of BIIB059 up to and including 20 mg/kg in healthy subjects have demonstrated acceptable tolerability. BDCA2 target engagement, as measured by BDCA2 internalization and reappearance, was observed in a dose-dependent manner across the dose range of 0.3 mg/kg to 20 mg/kg. EC90 values for BDCA2 internalization were derived from population-based PK and PD modeling with the mean value of 1.5 g/mL. IC90 for IFNα inhibition was estimated from in vitro to in vivo extrapolation of BDCA2 internalization and IFNα inhibition.

BIIB059 fixed doses of 50 mg, 150 mg and 450 mg subcutaneous (SC) administration every 4 weeks (Q4W) with an additional dose (“loading dose”) two weeks after administration of the first dose (Week 2) are supported by the following:
(1) The low dose of 50 mg SC Q4W was chosen to maintain BDCA2 internalization for the majority of the dosing interval.
(2) The middle dose of 150 mg SC Q4W was selected to achieve minimum observed concentration (Cmin) levels similar to the calculated IC90 for IFNα.
(3) The top dose of 450 mg SC Q4W was selected to achieve Cmin levels similar to 3-fold of the calculated IC90 for IFNα inhibition. Furthermore, this dose regimen with an additional dose of 450 mg at Week 2 and a bioavailability (F) of 0.45 is expected to result in cumulative exposure over 3 months comparable to that achieved by the single dose of 20 mg/kg IV for a 65-kg person, the highest dose tested in healthy volunteers.

To ensure sufficient drug exposure and concentration levels above the target steady-state values within 1 month following SC administration, a SC loading dose on Week 2 (Day 15—i.e., 15 days after administration of the first dose) will be included.

PK data using weight-adjusted dosing showed that body weight is not an influential covariate for BIIB059 exposure. Further, population PK simulations showed that both weight-adjusted dosing and fixed dosing result in comparable BIIB059 exposure. Fixed dose regimens, therefore, are reasonable.

A high dose of 450 mg SC Q4W (for 12 weeks) and a loading dose at Week 2 is based on PK simulations using data with both SC and IV Q2W regimens, and the expectation that the 450 mg dose level will have adequate target (BDCA2) coverage to suppress pDC function, including the production of type I IFN, over the 12 weeks.

### Example 6: Anti-BDCA2 High Concentration Formulation Study

An anti-BDCA2 antibody drug product was formulated at a concentration of 150 mg/mL in 20 mM histidine, 100 mM Arg·HCl, 3% sucrose, 0.05% polysorbate-80 at pH 5.5. To enable subcutaneous administration of anti-BDCA2 antibody at high doses, a formulation study was conducted to examine the stability of anti-BDCA2 antibody liquid formulations at concentrations above 150 mg/mL. Concentrations of 200, 225, and 240 mg/mL were examined in this study. Arginine and sucrose levels were also varied to understand the role of these excipients in the stability of high concentration formulations. Additionally, the pH of the formulations was increased to 6.0 or 6.5 to reduce the formation of basic species. A total of ten formulations was tested (see Table 6 for formulation compositions).

TABLE 6
Tested Formulations

| | [anti-BDCA2 | | | | | |
|---|---|---|---|---|---|---|
| | antibody] | Arg•HCl | Sucrose | | His | PS 80 |
| Formulation | (mg/mL) | (mM) | (%) | pH | (mM) | (%) |
| 1 | 200 | 250 | 3 | 6 | 20 | 0.05 |
| 2 | 200 | 100 | 3 | 6.5 | 20 | 0.05 |
| 3 | 200 | 250 | 3 | 6.5 | 20 | 0.05 |
| 4 | 225 | 100 | 3 | 6 | 20 | 0.05 |
| 5 | 225 | 250 | 1 | 6 | 20 | 0.05 |
| 6 | 225 | 250 | 1 | 6.5 | 20 | 0.05 |
| 7 | 240 | 100 | 1 | 6 | 20 | 0.05 |
| 8 | 240 | 250 | 1 | 6 | 20 | 0.05 |
| 9 | 240 | 100 | 1 | 6.5 | 20 | 0.05 |
| 10 | 240 | 250 | 1 | 6.5 | 20 | 0.05 |

All formulations were incubated at four conditions: (i) 5° C., (ii) 25° C./60% RH, (iii) 30° C./70% RH, and (iv) 40° C./75% RH. At predetermined time points, samples were pulled for analysis, which included size exclusion chromatography (SEC) for quantification of aggregates and imaging capillary isoelectric focusing (icIEF) for quantification of basic isoforms.

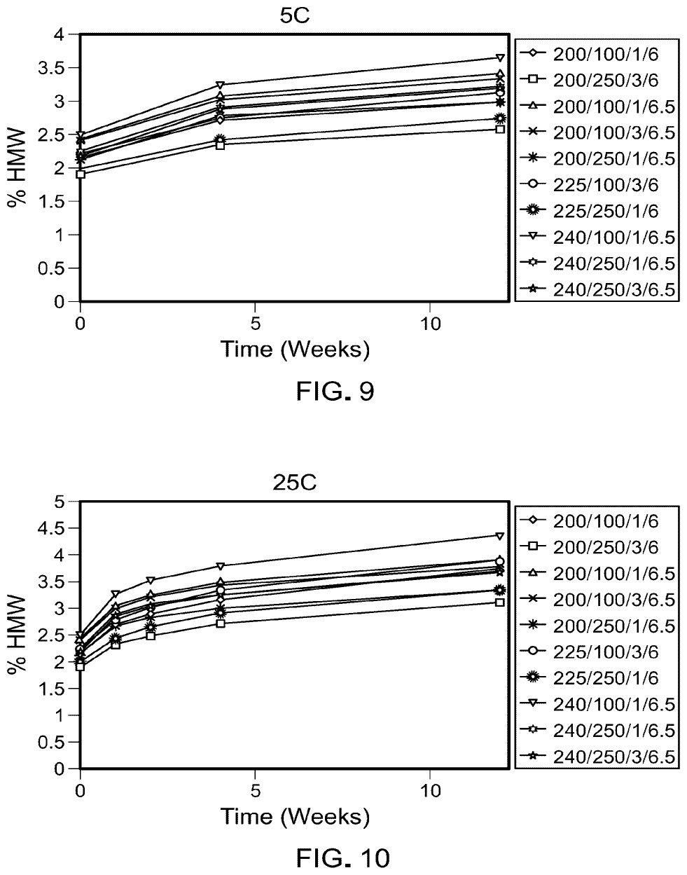

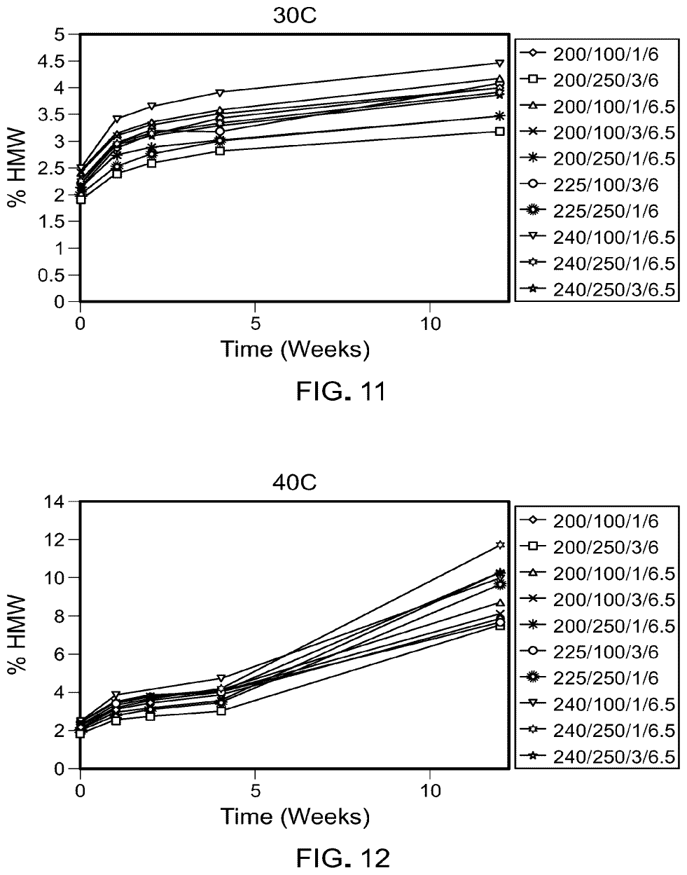

At 5° C., Formulations 1 and 5, labeled 200/250/3/6 and 225/250/1/6, respectively, had the lowest aggregation at all the tested time points (0, 4, and 12 weeks) (FIG. 9). At 25° C. (FIG. 10), 30° C. (FIG. 11), and 40° C. (FIG. 12), Formulation 1 consistently exhibited the lowest aggregate formation compared to other formulations. Thus, Formulation 1 was identified as the best performing formulation in this study. Linear modeling of the aggregation data from this study indicated that protein concentration, arginine concentration, and pH of the formulation significantly impacted aggregation.

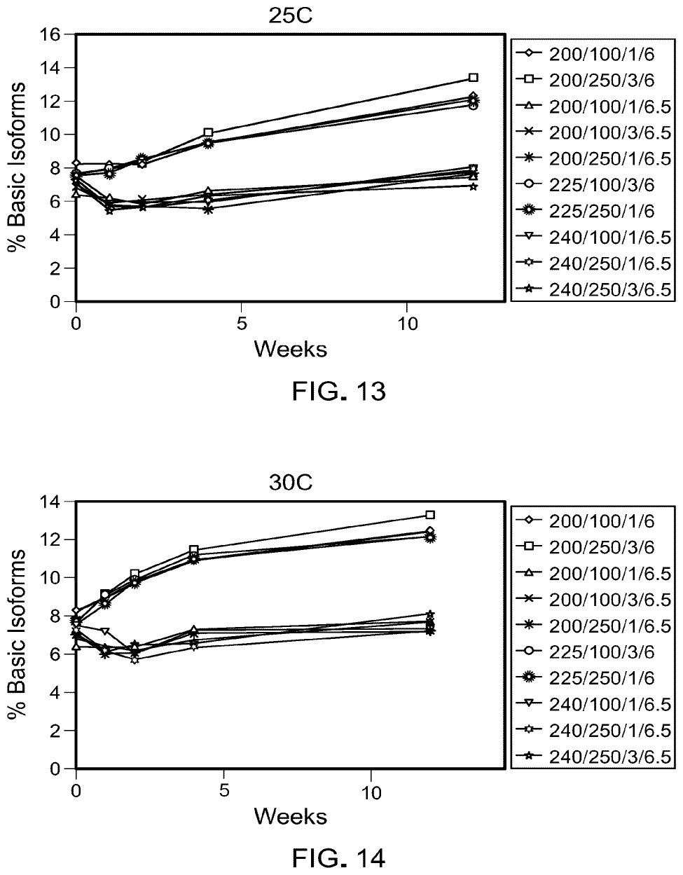

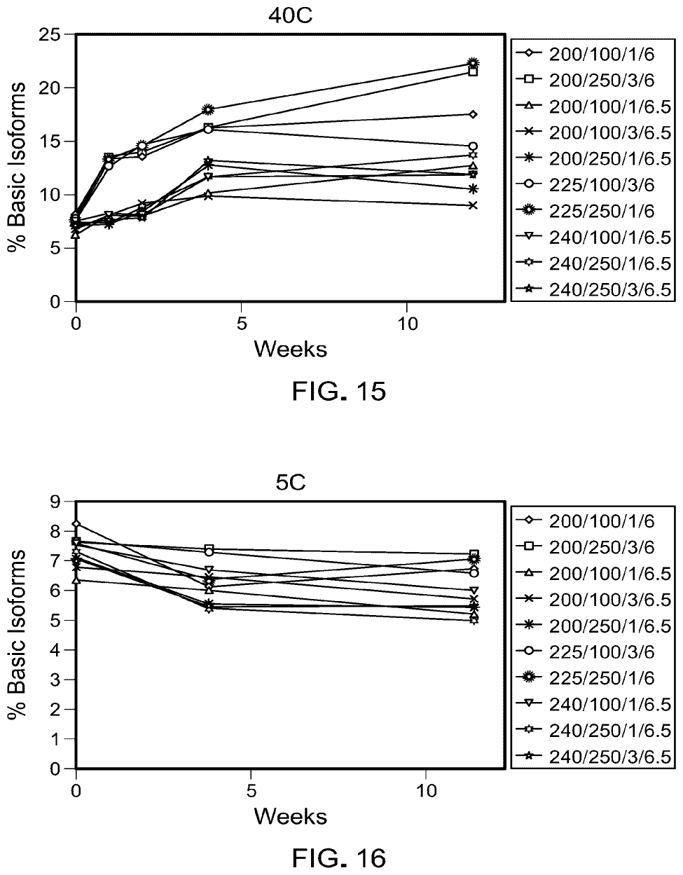

Basic species formation was highly dependent on pH: increased levels were seen in formulations at pH 6.0 compared to formulations at pH 6.5. This trend was particularly apparent at 25° C. (FIG. 13), 30° C. (FIG. 14), and 40° C. (FIG. 15). Formulations at pH 6.0 also tended to exhibit an increase in basic isoforms over time at 25° C. (FIG. 13), 30° C. (FIG. 14), and 40° C. (FIG. 15). At 5° C., there was no consistent increase in basic isoforms in any of the formulations (FIG. 16), unlike previous findings in the exemplary BDCA2 formulation (i.e., wherein the anti-BDCA2 drug product is formulated at a concentration of 150 mg/mL in 20 mM histidine, 100 mM Arg·HCl, 3% sucrose, 0.05% polysorbate-80 at pH 5.5).

### Example 7: Assessing Impact of Thiol-Containing Oxidizing Agent(s) in Anti-BDCA2 Antibody Formulations

### Materials and Methods

### Proteins and Reagents

Anti-BDCA2 antibody (BIIB059), SB4 (BENEPALI®), and anti-αvβ5 antibody (STX 200) were formulated according to the table below:

| | Conc. | | |
|---|---|---|---|
| Molecule | (mg/ml) | Buffer | pH |
| BDCA2 | 150 | 20 mM His, 100 mM Arg · HCl, | 5.5 |
| | | 3% Sucrose, 0.05% PS80 | |
| SB4 | 50 | 10 mM sodium phosphate, 140 mM NaCl, | 6.2 |
| | | 1% sucrose | |
| STX 200 | 50 | 20 mM Histidine, 5% Sorbitol, 0.05% PS80 | 6.5 |

Reduced and Oxidized forms of L-Glutathione (GSH and GSSG) were obtained from Sigma Aldrich (St. Louis, MO).

### Size Exclusion HPLC

Size exclusion HPLC (SEC) experiments were performed on a Waters Acquity UPLC instrument equipped with an Acquity UPLC BEH200 SEC analytical column coupled with a guard column. UV detection was performed at 280 nm. A sample amount of 20 μg was injected on to the column by a constant flow rate of 0.35 mL/min mobile phase. Each sample ran for 10 min.

### Stability Studies

SB4 and STX 200 were concentrated to 150 mg/ml in 10K centrifugal filters. Stock solutions of 20 mM GSH and 10 mM GSSG, prepared in corresponding formulation buffers, were spiked in protein solutions to achieve final concentrations of 0.4 mM and 0.2 mM, respectively. The prepared solutions were plated in WebSeal plates with glass inserts, sealed and incubated at 25° C./60% RH and 40° C./75% RH for 3 months. Analysis of % HMW was performed by SEC at predetermined time points.

### Results and Discussion

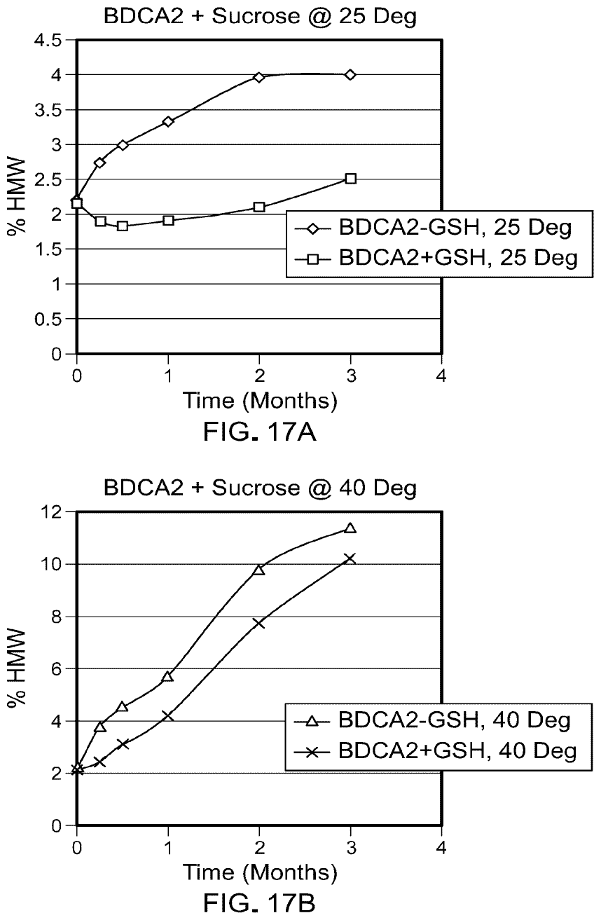

Glutathione, a tripeptide (γ-Glu-Cys-Gly) regulates disulfide bond formation. The reduced form (GSH) cleaves misbridged disulfide bonds and the oxidized form (GSSG) facilitates their formation. Hence, aggregated proteins incubated with the redox pair (i.e., GSH+GSSG) would refold to the correct native conformation and affect the aggregation kinetics. Anti-BDCA2 antibody in presence of glutathione shows an initial reversible aggregation followed by an aggregation rate that is slower to the formulation with no glutathione at 25° C. (FIG. 17, left panel). Higher temperatures (40° C.) add to the diversity of aggregation mechanisms, with conformational stability also coming into play (FIG. 17, right panel). Hence glutathione alone was not able to achieve similar reduction as at 25° C.

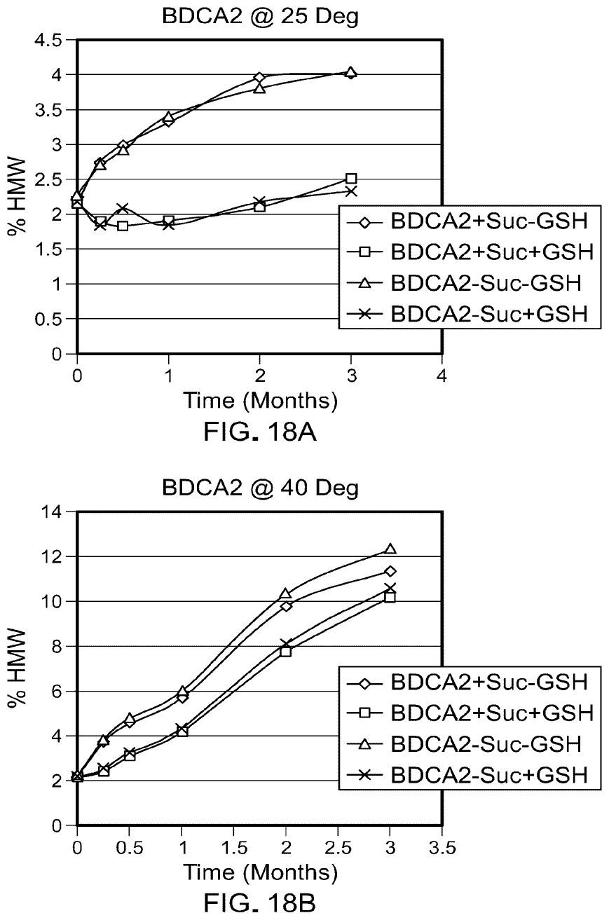

Sucrose is a widely used excipient for protein stabilization. It is preferentially excluded from the protein surface, thus favoring its native conformation. Absence of sucrose in the anti-BDCA2 antibody formulation did not affect the aggregation profile (FIG. 18), further emphasizing the role of disulfide bond scrambling to control aggregation in BDCA2.

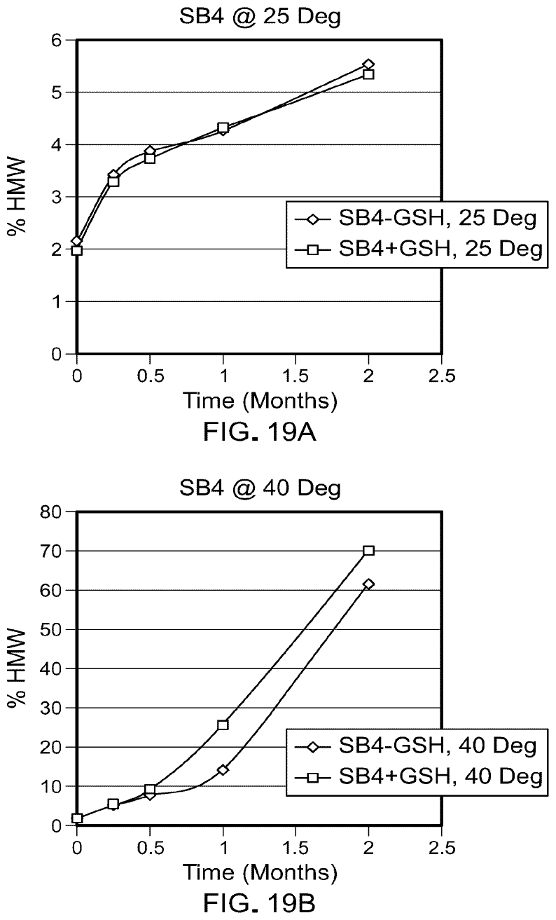

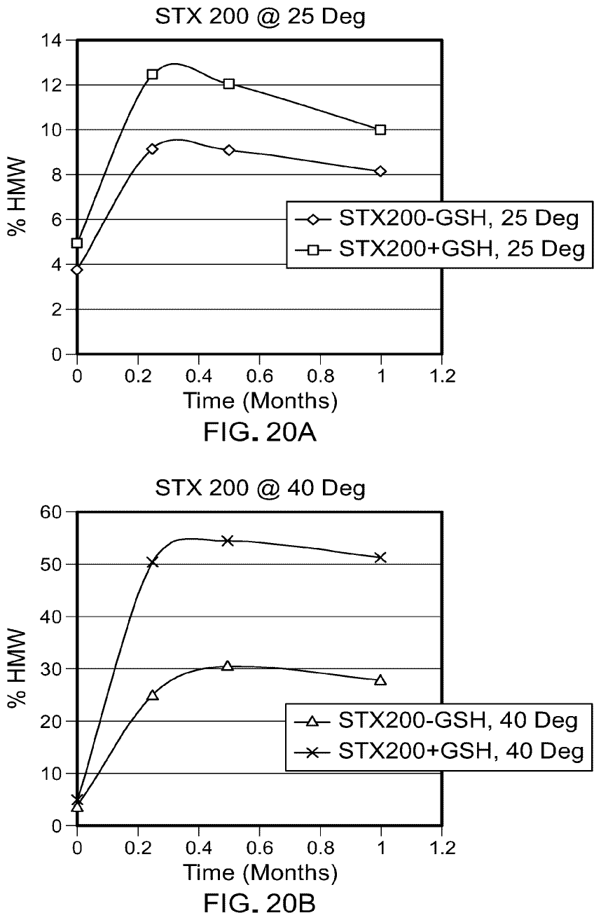

Addition of glutathione negatively impacts STX200, where an increase in aggregation was observed (FIG. 20). STX 200 is an aglycosylated molecule, demonstrating poor conformational stability at higher temperatures. Hence, unfolding of the molecule exposes the thiol group making it more susceptible to crosslinking with the thiol in glutathione and promoting further aggregation. Glutathione also did not have any effect on the aggregation kinetics in SB4, a fusion protein at 25° C., but facilitated faster aggregation at 40° C. (FIG. 19).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A pharmaceutical composition comprising an anti-Blood Dendritic Cell Antigen 2 (BDCA2) antibody or BDCA2-binding fragment thereof, sucrose, and arginine hydrochloride (Arg·HCl), wherein the anti-BDCA2 antibody or BDCA2-binding fragment thereof comprises an immunoglobulin heavy chain variable domain (VH) and an immunoglobulin light chain variable domain (VL), the VH and VL, respectively, comprising:
(a) VH complementarity determining regions (CDRs), wherein
H-CDR1 consists of the amino acid sequence set forth in SEQ ID NO:1 or 17;
H-CDR2 consists of the amino acid sequence set forth in SEQ ID NO:2; and
H-CDR3 consists of the amino acid sequence set forth in SEQ ID NO:3; and
(b) VL CDRs, wherein
L-CDR1 consists of the amino acid sequence set forth in SEQ ID NO:4;
L-CDR2 consists of the amino acid sequence set forth in SEQ ID NO: 5; and
L-CDR3 consists of the amino acid sequence set forth in SEQ ID NO:6, and
wherein the composition has a pH of 5.0 to 6.5.

2. The pharmaceutical composition of claim 1, wherein the composition comprises the anti-BDCA2 antibody or BDCA2-binding fragment thereof at a concentration of (i) 50 mg/ml to 225 mg/ml; (ii) 75 mg/ml to 225 mg/ml; (iii) 125 mg/ml to 175 mg/ml; or (iv) 150 mg/ml.

3.-4. (canceled)

5. The pharmaceutical composition of claim 1, wherein the composition comprises sucrose at a concentration of (i) 0.05% to 10%; (ii) 1% to 5%; or (iii) 3%.

6.-7. (canceled)

8. The pharmaceutical composition of claim 1, wherein the composition comprises Arg·HCl at a concentration of (i) 50 mM to 250 mM; (ii) 75 mM to 125 mM; or (iii) 100 mM.

9.-11. (canceled)

12. The pharmaceutical composition of claim 1, wherein the composition comprises PS80 at a concentration of (i) 0.01% to 0.1%; (ii) 0.03% to 0.08%; or (iii) 0.05%.

13.-15. (canceled)

16. The pharmaceutical composition of claim 1, wherein the composition comprises histidine at a concentration of (i) 5 mM to 50 mM; (ii) 15 mM to 25 mM; or (iii) 20 mM.

17.-18. (canceled)

19. The pharmaceutical composition of claim 1, wherein the composition has a pH of (i) 5.3 to 5.7; (ii) 5.5; (iii) 5.7; or (iv) 6.0.

20. (canceled)

21. The pharmaceutical composition of claim 1, comprising:
the anti-BDCA2 antibody or the BDCA2-binding fragment thereof at a concentration of 125 mg/ml to 175 mg/ml;
sucrose at a concentration of 1% to 5%;
histidine at a concentration of 15 mM to 25 mM;
Arg·HCl at a concentration of 75 mM to 125 mM; and
PS80 at a concentration of 0.03% to 0.08%,
wherein the composition has a pH of 5.3 to 5.7.

22. The pharmaceutical composition of claim 1, comprising:
the anti-BDCA2 antibody or the BDCA2-binding fragment thereof at a concentration of 150 mg/ml;
sucrose at a concentration of 3%;
histidine at a concentration of 20 mM;
Arg·HCl at a concentration of 100 mM; and
PS80 at a concentration of 0.05%,
wherein the composition has a pH of 5.7.

23. The pharmaceutical composition of claim 1, wherein:
(i) the VH consists of a sequence at least 80% identical to SEQ ID NO:7 and the VL consists of a sequence at least 80% identical to SEQ ID NO:8;
(ii) the VH consists of a sequence at least 90% identical to SEQ ID NO:7 and the VL consists of a sequence at least 90% identical to SEQ ID NO:8; or
(iii) the VH consists of the amino acid sequence set forth in SEQ ID NO:7 and the VL consists of the amino acid sequence set forth in SEQ ID NO:8.

24. The pharmaceutical composition of claim 1, wherein the anti-BDCA2 antibody comprises an immunoglobulin heavy chain and an immunoglobulin light chain, wherein:
(i) the heavy chain consists of a sequence at least 80% identical to SEQ ID NO:9 and the light chain consists of a sequence at least 80% identical to SEQ ID NO:10;
(ii) the heavy chain consists of a sequence at least 90% identical to SEQ ID NO:9 and the light chain consists of a sequence at least 90% identical to SEQ ID NO:10; or
(iii) the heavy chain consists of the amino acid sequence set forth in SEQ ID NO:9 and the light chain consists of the amino acid sequence set forth in SEQ ID NO: 10.

25. A method of treating a condition selected from the group consisting of systemic lupus erythematosus, cutaneous lupus erythematosus, discoid lupus erythematosus, Sjogren's syndrome, dermatopolymyositis, scleroderma, and cytokine release syndrome in a human subject in need thereof, the method comprising administering to the human subject the pharmaceutical composition of claim 21.

26. The method of claim 25, wherein the pharmaceutical composition is administered subcutaneously to the human subject.

27. The method of claim 25, wherein the anti-BDCA2 antibody or BDCA2-binding fragment thereof of the pharmaceutical composition is administered to the human subject at a dose of (i) 50 mg every four weeks; (ii) 150 mg every four weeks; or (iii) 450 mg every four weeks.

28.-30. (canceled)

31. The method of claim 27, wherein the human subject is administered a loading dose of the anti-BDCA2 antibody or BDCA2-binding fragment thereof two weeks after the first administration of the anti-BDCA2 antibody or BDCA2-binding fragment thereof, wherein the loading dose is 50 mg, 150 mg, or 450 mg.

32.-38. (canceled)

39. The method of claim 27, wherein the human subject is administered at least 4 doses, at least 7 doses, or at least 10 doses of the anti-BDCA2 antibody or antigen-binding fragment thereof.

40.-41. (canceled)

42. The method of claim 27, wherein:
(i) the VH consists of a sequence at least 80% identical to SEQ ID NO:7 and the VL consists of a sequence at least 80% identical to SEQ ID NO:8;
(ii) the VH consists of a sequence at least 90% identical to SEQ ID NO:7 and the VL consists of a sequence at least 90% identical to SEQ ID NO:8; or
(iii) the VH consists of the amino acid sequence set forth in SEQ ID NO:7 and the VL consists of the amino acid sequence set forth in SEQ ID NO:8.

43.-52. (canceled)

53. A syringe, injector, or pump comprising a sterile preparation of an anti-BDCA2 antibody or BDCA2-binding fragment thereof, wherein the syringe, injector, or pump is adapted for subcutaneous administration of the anti-BDCA2 antibody or BDCA2-binding fragment thereof at a fixed dose of 50 mg, 150 mg, or 450 mg, and wherein the anti-BDCA2 antibody or BDCA2-binding fragment thereof comprises an immunoglobulin heavy chain variable domain (VH) and an immunoglobulin light chain variable domain (VL), the VH and VL, respectively, comprising:
(a) VH complementarity determining regions (CDRs), wherein
H-CDR1 consists of the amino acid sequence set forth in SEQ ID NO:1;
H-CDR2 consists of the amino acid sequence set forth in SEQ ID NO:2; and
H-CDR3 consists of the amino acid sequence set forth in SEQ ID NO:3; and
(b) VL CDRs, wherein
L-CDR1 consists of the amino acid sequence set forth in SEQ ID NO:4;
L-CDR2 consists of the amino acid sequence set forth in SEQ ID NO: 5; and
L-CDR3 consists of the amino acid sequence set forth in SEQ ID NO:6.

54.-80. (canceled)

81. The pharmaceutical composition of claim 1, wherein the composition comprises a thiol-containing antioxidant.

82. The pharmaceutical composition of claim 81, wherein the thiol-containing antioxidant is selected from the group consisting of GSH, GSSG, the combination of GSH and GSSG, cystine, cysteine, and the combination of cysteine and cystine.

83.-85. (canceled)

86. The pharmaceutical composition of claim 81, wherein the thiol-containing antioxidant is at a concentration of (i) 0.02 mM to 2 mM; (ii) 0.2 mM; (iii) 0.4 mM; or (iv) 1 mM.

87.-90. (canceled)

91. A pharmaceutical composition comprising an anti-Blood Dendritic Cell Antigen 2 (BDCA2) antibody or BDCA2-binding fragment thereof and:
histidine at a concentration of 10 mM to 30 mM;
Arg·HCl at a concentration of 50 mM to 250 mM; and
PS80 at a concentration of 0.02% to 0.08%,
wherein the composition has a pH of 5.0 to 6.5, and
wherein the anti-BDCA2 antibody or BDCA2-binding fragment thereof comprises an immunoglobulin heavy chain variable domain (VH) and an immunoglobulin light chain variable domain (VL), the VH and VL, respectively, comprising:
(a) VH complementarity determining regions (CDRs), wherein
H-CDR1 consists of the amino acid sequence set forth in SEQ ID NO:1 or 17;
H-CDR2 consists of the amino acid sequence set forth in SEQ ID NO:2; and
H-CDR3 consists of the amino acid sequence set forth in SEQ ID NO:3; and
(b) VL CDRs, wherein
L-CDR1 consists of the amino acid sequence set forth in SEQ ID NO:4;
L-CDR2 consists of the amino acid sequence set forth in SEQ ID NO: 5; and
L-CDR3 consists of the amino acid sequence set forth in SEQ ID NO:6.

92. The pharmaceutical composition of claim 91, comprising the anti-BDCA2 antibody or the BDCA2-binding fragment thereof at a concentration of 50 mg/ml to 225 mg/ml.

93. The pharmaceutical composition of claim 91, comprising sucrose at a concentration of 1% to 10%.

94. The pharmaceutical composition of claim 91, comprising a thiol-containing antioxidant.

95. The pharmaceutical composition of claim 94, wherein the thiol-containing antioxidant is selected from the group consisting of GSH, GSSG, the combination of GSH and GSSG, cystine, cysteine, and the combination of cysteine and cystine.

96.-103. (canceled)

104. The pharmaceutical composition of claim 91, comprising:
the anti-BDCA2 antibody or the BDCA2-binding fragment thereof at a concentration of 150 mg/ml;
sucrose at a concentration of 3%;
histidine at a concentration of 20 mM;
Arg·HCl at a concentration of 100 mM;
PS80 at a concentration of 0.05%; and
GSH at a concentration of 0.4 mM, or GSSG at a concentration of 0.2 mM, or GSH at a concentration of 0.4 mM and GSSG at a concentration of 0.2 mM,
wherein the composition has a pH of 5.7.

105.-106. (canceled)

107. A pharmaceutical composition comprising:
(i) an anti-BDCA2 antibody or the BDCA2-binding fragment thereof at a concentration of 200 mg/ml;
sucrose at a concentration of 3%;
histidine at a concentration of 20 mM;
Arg·HCl at a concentration of 250 mM;
PS80 at a concentration of 0.05%; and
wherein the composition has a pH of 6.0, or
(ii) an anti-BDCA2 antibody or the BDCA2-binding fragment thereof at a concentration of 225 mg/ml;
sucrose at a concentration of 1%;
histidine at a concentration of 20 mM;
Arg·HCl at a concentration of 250 mM;
PS80 at a concentration of 0.05%; and
wherein the composition has a pH of 6.0; and
wherein the anti-BDCA2 antibody or BDCA2-binding fragment thereof comprises an immunoglobulin heavy chain variable domain (VH) and an immunoglobulin light chain variable domain (VL), the VH and VL, respectively, comprising:
(a) VH complementarity determining regions (CDRs), wherein
H-CDR1 consists of the amino acid sequence set forth in SEQ ID NO:1 or 17;
H-CDR2 consists of the amino acid sequence set forth in SEQ ID NO:2; and
H-CDR3 consists of the amino acid sequence set forth in SEQ ID NO:3; and
(b) VL CDRs, wherein
L-CDR1 consists of the amino acid sequence set forth in SEQ ID NO:4;
L-CDR2 consists of the amino acid sequence set forth in SEQ ID NO:5; and
L-CDR3 consists of the amino acid sequence set forth in SEQ ID NO:6.

108. (canceled)

109. The pharmaceutical composition of claim 107, comprising a thiol-containing antioxidant.

110.-113. (canceled)

114. A method of treating a condition selected from the group consisting of systemic lupus erythematosus, cutaneous lupus erythematosus, discoid lupus erythematosus, Sjogren's syndrome, dermatopolymyositis, scleroderma, and cytokine release syndrome in a human subject in need thereof, the method comprising administering to the human subject the pharmaceutical composition of claim 104.

115.-118. (canceled)

119. The method of claim 114, wherein the anti-BDCA2 antibody or BDCA2-binding fragment thereof of the pharmaceutical composition is administered to the human subject
at the dose corresponding to the human subject's weight as recited below:

| Weight | Dose |
|---|---|
| 10 to 18 kg | 18 mg every four weeks |
| 18.1 to 25 kg | 22 mg every four weeks |
| 25.1 to 48 kg | 28 mg every four weeks |
| greater than 48 kg | 50 mg every four weeks; or |
| Weight | | Dose | |
|---|---|---|---|
| 10 to 18 | kg | 40 | mg every four weeks |
| 18.1 to 25 | kg | 56 | mg every four weeks |
| 25.1 to 48 | kg | 80 | mg every four weeks |
| greater than 48 | kg | 150 | mg every four weeks. |

120.-123. (canceled)

124. The method of claim 119, wherein:
(i) the VH consists of a sequence at least 80% identical to SEQ ID NO:7 and the VL consists of a sequence at least 80% identical to SEQ ID NO:8;
(ii) the VH consists of a sequence at least 90% identical to SEQ ID NO:7 and the VL consists of a sequence at least 90% identical to SEQ ID NO:8; or
(iii) the VH consists of the amino acid sequence set forth in SEQ ID NO:7 and the VL consists of the amino acid sequence set forth in SEQ ID NO:8.

125. (canceled)

126. A syringe, injector, or pump comprising a sterile preparation of the pharmaceutical composition of claim 104 adapted for subcutaneous administration of the anti-BDCA2 antibody or BDCA2-binding fragment thereof at a fixed dose of 18 mg, 22 mg, 28 mg, 40 mg, 50 mg, 56 mg, 80 mg, 150 mg, or 450 mg.

127. (canceled)

128. The syringe, injector, or pump of claim 126, wherein:
(i) the VH consists of a sequence at least 80% identical to SEQ ID NO:7 and the VL consists of a sequence at least 80% identical to SEQ ID NO:8;
(ii) the VH consists of a sequence at least 90% identical to SEQ ID NO:7 and the VL consists of a sequence at least 90% identical to SEQ ID NO:8; or
(iii) the VH consists of the amino acid sequence set forth in SEQ ID NO:7 and the VL consists of the amino acid sequence set forth in SEQ ID NO:8.

129. (canceled)
